(19) United States
(12) Patent Application Publication
(10) Pub. No.: US 2026/0204375 A1
(43) Pub. Date: Jul. 16, 2026

(54) PATHWAY-BASED HEALTH OPTIMIZATION

(71) Applicant: GNQ INSILICO, INC., PLEASANTON, CA (US)

(72) Inventors: SUDHIR SAXENA, PLEASANTON, CA (US); SARWAT ANWER, PLEASANTON, CA (US)

(21) Appl. No.: 19/449,913

(22) Filed: Jan. 15, 2026

Related U.S. Application Data
(60) Provisional application No. 63/746,036, filed on Jan. 16, 2025.

(51) Int. Cl. G16H 20/00; G16H 50/30

(52) U.S. Cl. CPC G16H 20/00; G16H 50/30

(57) ABSTRACT

Generating one or more health-related recommendations including processing one or more autonomously integrated biologically-related data sets corresponding to a patient; generating a virtual representation of the patient via one or more biological pathways based on the one or more biologically-related data sets; analyzing the one or more biologically-related data sets using the virtual representation of the patient; and generating and displaying, based on the analysis, one or more health-related recommendations associated with the patient.

### CROSS-REFERENCE TO RELATED APPLICATION

This application claims the priority benefit under 35 U.S.C. § 119(e) of U.S. Provisional Application No. 63/746,036, filed on January 16, 2025, the disclosure of which is incorporated by reference in its entirety as if fully set forth herein.

### FIELD

The field of the present disclosure relates to health data processing and computational health optimization systems. More specifically, the disclosure relates to systems and methods for computational analysis and modeling of a digital twin used to support health-related decision making and optimization.

### BACKGROUND

The statements in this section merely provide background information related to the present disclosure and may not constitute prior art.

Healthcare computing systems deployed across clinical, research, and personal health environments process biologically related data sets to support assessment, monitoring, and therapeutic decision making for patients. Many existing implementations rely on fixed analytical models, predefined metrics, or modality-specific pipelines that evaluate clinical records, imaging data, physiological measurements, or molecular data in isolation. Conventional approaches often emphasize aggregated indicators or discrete measurements without accounting for how biologically related data sets interact across biological pathways or evolve over time. As patient data streams expand to include longitudinal records, multi-omics measurements, and real-time physiological inputs, existing systems lack a unified analytical framework capable of contextualizing such data within biologically meaningful structures.

Current computational frameworks for healthcare analysis do not fully capture the complexity of biological systems or the dynamic interplay between molecular, physiological, and temporal factors that influence patient health states. Many implementations rely on static models or correlation-based techniques that do not adapt to biological variation or evolving pathway interactions across individual patients. Data derived from molecular profiling, clinical observation, and physiological monitoring often remains siloed across separate analytical stages, limiting the ability to derive predictive or integrative insights at a pathway level. The present disclosure addresses these and other issues related to providing a means for adaptive and data-driven management of physiological processes through continuous analysis, modeling, and optimization of patient-specific biological states.

### SUMMARY

According to embodiments of the present disclosure, various systems, methods, and computer program products for pathway-based health optimization are described herein. In various aspects, a computer-implemented method utilized to support an implementation of the pathway-based health optimization includes processing, by a computing device, one or more autonomously integrated biologically-related data sets corresponding to a patient; generating, by the computing device and in response to processing the one or more autonomously integrated biologically-related data sets, a virtual representation of the patient via one or more biological pathways; and displaying, by the computing device, one or more health-related recommendations generated based on an analysis of the one or more biologically-related data sets using the virtual representation of the patient. In various aspects, a system includes a memory and one or more processing devices operatively coupled to the memory, where the one or more processing devices perform operations corresponding to the method steps described herein. In various aspects, a computer program product includes a non-transitory computer-readable medium storing processor-executable instructions that, when executed by at least one processor, cause the at least one processor to perform the computer-implemented methods described herein.

Although embodiments are described in the context of pathway-based analysis of biologically-related data sets for health optimization, the described computational framework may be applied to other contexts in which integrated data sets are analyzed to generate representations and recommendations associated with complex systems, including other physiological domains or data-driven optimization environments.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

In order that the disclosure may be well understood, there will now be described various forms thereof, given by way of example, reference being made to the accompanying drawings, in which:

FIG. 1 illustrates a block diagram of an example computing system configured to process biologically-related data sets and optimize health-related recommendations associated with a patient according to embodiments of the present disclosure.

FIG. 2 illustrates a block diagram of an example system architecture utilized to process biologically-related data sets and optimize health-related recommendations associated with a patient according to embodiments of the present disclosure.

FIG. 3 sets forth a flowchart of an example method for pathway-based health optimization in accordance with embodiments of the present disclosure.

FIG. 4 sets forth a flowchart of an example method for analyzing biological variation and identifying spatiotemporal patterns within biologically-related data sets in accordance with embodiments of the present disclosure.

FIG. 5 sets forth a flowchart of an example method for conducting predictive risk assessment using biologically-related data sets and a pathway-based virtual representation of a patient in accordance with embodiments of the present disclosure.

FIG. 6 sets forth a flowchart of an example method for simulating one or more safety and efficacy models associated with health-related recommendations in accordance with embodiments of the present disclosure.

FIG. 7 sets forth a flowchart of an example method for iteratively refining one or more strategies for adjusting biologically-related solutions corresponding to health-related recommendations in accordance with embodiments of the present disclosure.

FIG. 8 sets forth a flowchart of an example method for executing real-time biological health state modeling in accordance with embodiments of the present disclosure.

FIG. 9 sets forth a flowchart of an example method for providing internal anatomical visualization capabilities and external form visualization capabilities to a user in accordance with embodiments of the present disclosure.

FIG. 10 illustrates a block diagram of a cloud service provider architecture in accordance with some embodiments of the present disclosure.

FIG. 11 illustrates a block diagram of an electronic device in a network environment according to embodiments of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

Optimization of patient health using biologically-related data sets in clinical, research, and personal health environments remains constrained by systems that rely on static analytical models and fragmented data processing workflows. Many existing approaches evaluate clinical records, physiological measurements, imaging data, or molecular information as isolated inputs, without accounting for how such biologically-related data sets interact across biological pathways or evolve over time. As a result, current systems provide limited capability to contextualize patient data within pathway-level biological relationships or to adapt health-related analysis as patient conditions change. These limitations are further amplified by distributed data ecosystems in which longitudinal records, multi-omics measurements, and real-time physiological inputs are not integrated within a unified computational framework, reducing the consistency and adaptability of health-related recommendations.

To address such challenges, the present disclosure sets forth various systems and methods of pathway-based health optimization. The described approaches process one or more autonomously integrated biologically-related data sets corresponding to a patient to generate a virtual representation of the patient via one or more biological pathways, and to analyze the biologically-related data sets using the virtual representation to generate health-related recommendations. In various embodiments, the described systems support biological variation analysis, identification of spatiotemporal patterns, predictive risk assessment, safety and efficacy modeling, iterative refinement of biologically-related strategies, and real-time biological health state modeling. Benefits provided include, but are not limited to, improved contextualization of patient data at a pathway level, increased adaptability to biological variation and temporal change, enhanced scalability across diverse data sources, and more consistent generation of health-related recommendations grounded in integrated biological analysis.

In one or more embodiments, the one or more health-related recommendations correspond to autonomously optimized biologically-related solutions that are evidence-based and personalized relative to the patient, and that are optimized in relation to one or more existing protocols, one or more novel protocols, one or more clinical trial enrollment opportunities, personalized treatment protocols, preventative intervention strategies, or a combination thereof.

As used herein, the term “biologically-related data sets” refers to one or more data sets associated with biological, physiological, clinical, or health-related characteristics of a patient. Biologically-related data sets may include, without limitation, clinical records, imaging data, molecular data, multi-omics data, physiological measurements, laboratory results, sensor-derived data, or combinations thereof. Biologically-related data sets may be obtained from disparate sources, may be structured or unstructured, and may be received as static data, periodically updated data, or continuously streaming data.

As used herein, the term “health-related recommendation” refers to an output generated by a computing system that is associated with a health-related assessment, analysis, prioritization, or guidance. A health-related recommendation may include informational outputs, ranked options, alerts, assessments, or suggested actions derived from analysis of biologically-related data sets. A health-related recommendation does not require autonomous medical decision making and may be provided for review, interpretation, or consideration by a user.

As used herein, the term “pathway-based” refers to the organization, analysis, modeling, or representation of biological information using one or more biological pathways as an analytical or structural framework. Pathway-based processing does not require the use of a specific pathway database, ontology, or modeling technique, and may include representations that capture functional, regulatory, metabolic, signaling, or other biological relationships among biological entities.

As used herein, the term “biological pathway” refers to a representation of biological relationships, interactions, or processes associated with biological function. A biological pathway may represent molecular interactions, cellular processes, metabolic activity, signaling cascades, regulatory mechanisms, or combinations thereof. Biological pathways may be defined explicitly or implicitly and may be derived from curated knowledge sources, computational inference, empirical data, or combinations thereof.

As used herein, the term “autonomously” refers to an operation performed by a computing system without requiring continuous manual intervention by a user. Autonomous operation may include execution based on predefined rules, learned parameters, policies, or system logic, and does not preclude initial configuration, supervision, constraints, or override by a user or external system.

As used herein, the term “digital twin” refers to a computational representation of a patient that reflects biological state using biologically-related data sets. A digital twin may model biological pathways, physiological characteristics, anatomical features, or functional relationships associated with the patient. A digital twin does not require exact replication of a patient and may represent biological state at varying levels of abstraction, resolution, or completeness.

As used herein, the term “virtual representation” refers to a computer-generated representation of a patient that is derived from one or more biologically-related data sets and corresponds to a digital twin of the patient, wherein the digital twin represents biological state through computational modeling. A virtual representation may be generated by processing integrated biological data to reflect biological pathways, anatomical structures, functional relationships, or combinations thereof associated with the patient. The virtual representation of the patient may include, without limitation, graphical depictions of internal anatomical features, external form characteristics, pathway-based models, or other visual constructs that convey biological state or variation as represented by the digital twin. The virtual representation may be updated based on newly received biologically-related data sets and may be used to support analysis, interpretation, and presentation of health-related recommendations associated with the patient.

In some embodiments, and as will be further described herein, analysis of the biologically-related data sets and generation of the virtual representation may further include causal inference techniques configured to distinguish causal biological relationships from statistical correlations. Such causal inference techniques may be used to identify cause-and-effect relationships among biological pathways, genetic influences, physiological processes, or environmental factors associated with the patient. The causal inference techniques may support evaluation of counterfactual scenarios, intervention impact estimation, or identification of upstream biological drivers that influence downstream pathway behavior, without limiting the virtual representation to correlation-based modeling.

In other embodiments, the causal inference techniques may further support comparison of multiple hypothetical interventions or alternative biologically-related strategies by evaluating counterfactual pathway responses within the virtual representation. Such comparison may include simulating how different candidate interventions, parameter adjustments, or pathway perturbations would influence biological state, risk, or outcome trajectories associated with the patient. The virtual representation may therefore be used to compare, rank, or prioritize alternative health-related recommendations based on predicted pathway-level effects, without requiring actual implementation of the compared interventions.

In further embodiments, the virtual representation may maintain multiple concurrent projected future biological states corresponding to different hypothetical scenarios, intervention strategies, or parameter configurations. Each projected future state may represent a distinct pathway-level trajectory derived from simulation, predictive risk assessment, or causal inference analysis. The system described herein may evaluate, compare, or rank such projected future states in parallel to assess relative outcomes, risk profiles, or biological tradeoffs, thereby enabling scenario branching and forward-looking evaluation of alternative health-related strategies prior to selection or presentation.

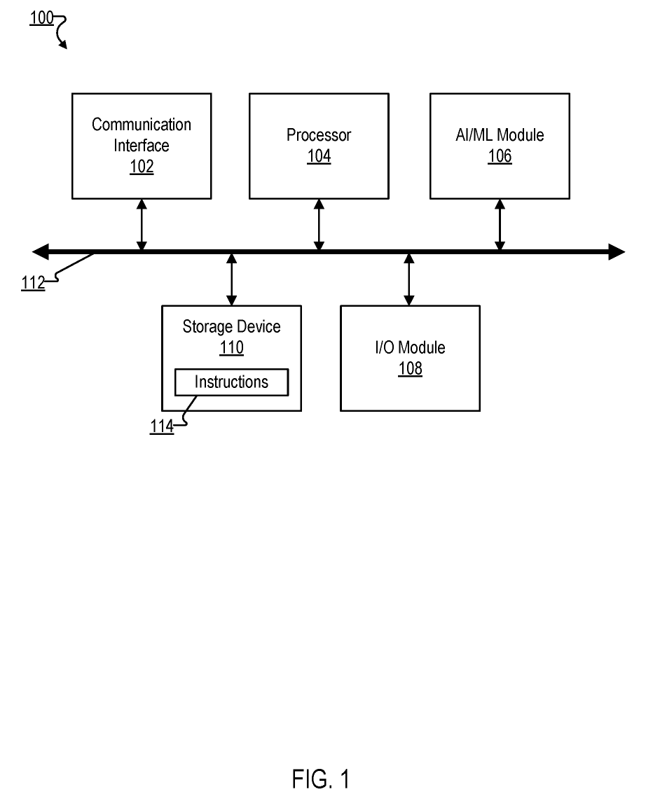

Example methods, systems, and products for pathway-based health optimization in accordance with embodiments of the present disclosure are described with reference to the accompanying drawings, beginning with FIG. 1. In one or more embodiments, FIG. 1 illustrates an example computing system 100 that may be specifically configured to perform one or more of the processes described herein associated with processing biologically-related data sets and optimizing health-related recommendations associated with a patient. As shown in FIG. 1, the computing system 100 may include a communication interface 102, a processor 104, an artificial intelligence and machine learning (AI/ML) module 106, an input and output (I/O) module 108, and a storage device 110 configured to store computer-executable instructions 114. The communication interface 102, the processor 104, the AI/ML module 106, the I/O module 108, and the storage device 110 are communicatively connected to one another via a communication infrastructure 112. The computer-executable instructions 114, when executed by the processor 104, may cause the computing system 100 to perform operations associated with processing one or more autonomously integrated biologically-related data sets corresponding to a patient, generating a virtual representation of the patient via one or more biological pathways, and generating and presenting health-related recommendations based on analysis of the biologically-related data sets using the virtual representation. While an exemplary computing system 100 is shown in FIG. 1, the illustrated components are not intended to be limiting, and additional or alternative components may be used in other embodiments. Components of the computing system 100 shown in FIG. 1 will now be described in additional detail.

The communication interface 102 may be configured to communicate with one or more external systems that can provide biologically-related data sets to, or receive generated output data from, the computing system 100. For example, the communication interface 102 may enable access to clinical record systems, imaging repositories, molecular or multi-omics data sources, physiological monitoring devices, and other patient-associated data repositories. As another example, the communication interface 102 may also enable interfaces that can distribute health-related recommendations, visualization data, or analytical outputs to client devices, clinical dashboards, or network-accessible platforms. Examples of the communication interface 102 can include, without limitation, a wired or wireless network interface, a high-speed interconnect, an application programming interface (API) gateway, or another communication module configured for data exchange between computing nodes in local, distributed, or cloud-based computational environments. In some embodiments, the communication interface 102 may include encryption, authentication, or other security mechanisms to support secure transmission of sensitive biological and health-related data across networked computing environments.

The processor 104 generally represents one or more processing units capable of executing operations of the computing system 100 associated with pathway-based health optimization. The processor 104 may execute the computer-executable instructions 114 stored in the storage device 110 to perform operations including processing one or more autonomously integrated biologically-related data sets corresponding to a patient, generating a virtual representation of the patient via one or more biological pathways, and generating health-related recommendations based on an analysis of the biologically-related data sets using the virtual representation. The processor 104 may include one or more general-purpose processing units, graphics processing units (GPUs), tensor processing units, or other processing resources configured to perform data-intensive computation, pathway-based analysis, and optimization routines. In some embodiments, the processor 104 may coordinate distributed processing across multiple computing nodes or cloud-based instances to support scalable data ingestion, pathway-level analysis, and low-latency generation of health-related recommendations across local, distributed, or cloud-based computing environments.

The AI/ML module 106 may be configured to perform adaptive modeling and analytical operations that support pathway-based health optimization. The AI/ML module 106 may receive data processed by the processor 104, including biologically-related data sets and pathway-based representations associated with a patient. Using the processed data, the AI/ML module 106 may support construction, evaluation, and refinement of the virtual representation of the patient via one or more biological pathways. In one or more embodiments, the AI/ML module 106 may employ machine learning models, graph-based analytical techniques, statistical learning processes, or optimization routines to capture relationships among biologically-related data sets and to support pathway-level analysis. When operating in coordination with the processor 104, the AI/ML module 106 may generate intermediate analytical outputs, update internal model parameters, and support generation of health-related recommendations based on an analysis of the biologically-related data sets using the virtual representation of the patient.

The I/O module 108 may include one or more input and output devices configured to receive user input and present output data generated by the computing system 100. The I/O module 108 may include any suitable combination of hardware, firmware, and software that supports data entry, configuration, and visualization capabilities. For input, the I/O module 108 may include interfaces for receiving biologically-related data sets, configuring processing parameters, and selecting operational modes associated with data integration, pathway-based analysis, or generation of health-related recommendations. Input devices may include keyboards, touchscreens, configuration panels, or web-based interfaces accessible to clinicians, researchers, or system administrators. For output, the I/O module 108 may include displays, dashboards, or graphical user interfaces (GUIs) configured to present health-related recommendations, pathway-based representations, analytical results, or system status information. In some embodiments, the I/O module 108 may further include interfaces for exporting processed data or recommendations to external systems, applications, or services for further analysis, visualization, or downstream processing.

The storage device 110 may include one or more types of non-volatile storage media and may be configured to store the computer-executable instructions 114 along with biologically-related data sets and other data generated or used by the computing system 100. The storage device 110 may maintain patient-associated data including clinical records, imaging data, molecular or multi-omics data, and other biologically-related data sets, as well as intermediate computational results such as pathway-based representations, analytical outputs, and recommendation data. In some embodiments, the storage device 110 may include structured data repositories or databases that can organize and index patient data, historical analysis results, and generated health-related recommendations to support efficient retrieval and reuse. The storage device 110 may further store logs or records of system operations, including data processing events and analytical outputs, to support traceability, auditing, and system monitoring. In certain embodiments, the storage device 110 may integrate with distributed or cloud-based storage resources to enable scalable data management across local, distributed, or networked computing environments.

The communication infrastructure 112 represents the internal interconnect architecture that links the communication interface 102, the processor 104, the AI/ML module 106, the I/O module 108, and the storage device 110 within the computing system 100. The communication infrastructure 112 supports the exchange of biologically-related data sets, pathway-based representations, intermediate analytical results, and health-related recommendations between components of the computing system 100. In some embodiments, the communication infrastructure 112 may be implemented using one or more buses, fabrics, or network topologies configured to provide sufficient throughput and latency characteristics to support coordinated data processing, pathway-based analysis, and generation of health-related recommendations across the computing system 100.

The computer-executable instructions 114 stored in the storage device 110 may define operations that, when executed by the processor 104, enable the computing system 100 to perform one or more processes associated with pathway-based health optimization. The computer-executable instructions 114 may include routines for receiving and processing biologically-related data sets, generating a virtual representation of a patient via one or more biological pathways, analyzing the biologically-related data sets using the virtual representation, and generating health-related recommendations based on the analysis. In some embodiments, the computer-executable instructions 114 may further include algorithms for performing biological variation analysis, identifying spatiotemporal patterns, conducting predictive risk assessments, simulating safety and efficacy models, and iteratively refining strategies associated with health-related recommendations. When executed, the computer-executable instructions 114 can coordinate operation of the communication interface 102, the processor 104, the AI/ML module 106, the I/O module 108, and the storage device 110 to enable the computing system 100 to function as an integrated platform for processing biologically-related data sets and generating health-related recommendations grounded in pathway-based analysis.

By combining these components, the computing system 100 is configured to execute the processes described herein for pathway-based health optimization. In particular, the computing system 100 supports operations including processing one or more autonomously integrated biologically-related data sets corresponding to a patient, generating a virtual representation of the patient via one or more biological pathways, analyzing the biologically-related data sets using the virtual representation, and generating health-related recommendations based on the analysis to be displayed to a user, such as clinicians, patients, and authorized healthcare personnel. The integrated architecture of the computing system 100 enables coordinated data ingestion, pathway-based analysis, and recommendation generation across local, distributed, or cloud-based computing environments. Through coordinated operation of the hardware and software components described herein, the computing system 100 provides a scalable and adaptable platform capable of supporting patient-specific health-related analysis and recommendation generation in accordance with embodiments of the present disclosure.

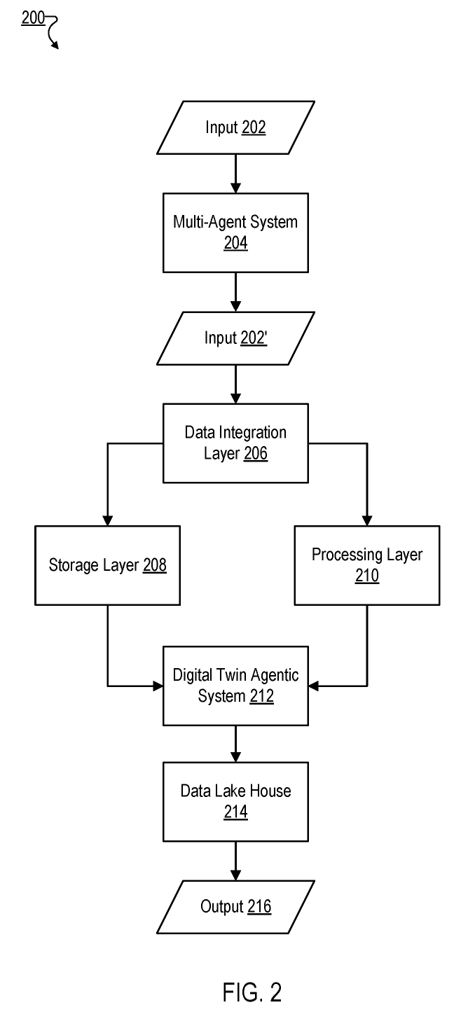

FIG. 1 therefore illustrates an example computing system 100 configured to perform operations associated with pathway-based health optimization through the processing of biologically-related data sets and pathway-based analysis. The described configuration enables the computing system 100 to acquire biologically-related data sets from multiple external sources, generate a virtual representation of a patient via one or more biological pathways, and generate health-related recommendations based on an analysis of the biologically-related data sets using the virtual representation. The modular design of the computing system 100 allows deployment within local computing environments, cloud-based platforms, or hybrid architectures, supporting scalability across individual and population-level use cases. The computing system 100 thus serves as a foundational architecture for executing the systems and methods described in the subsequent figures. FIG. 2 illustrates an example system architecture that builds upon the computing system 100 and further defines functional layers and data flow for processing biologically-related data sets and optimizing health-related recommendations in accordance with embodiments of the present disclosure.

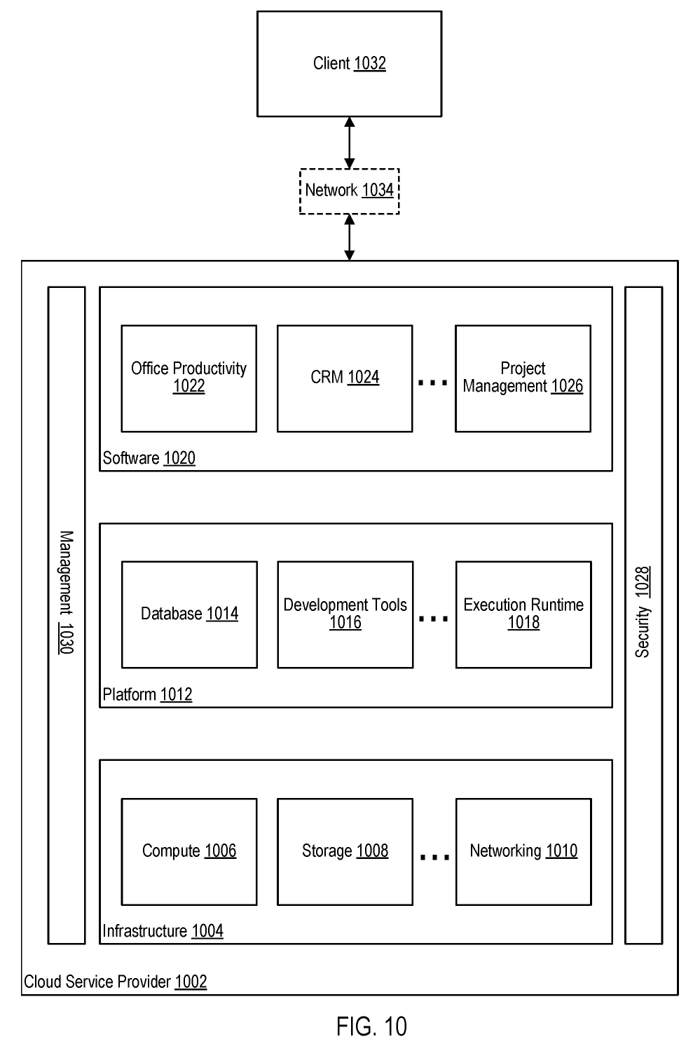

For further explanation, FIG. 2 sets forth a block diagram of an example system 200 configured for pathway-based health optimization in accordance with embodiments of the present disclosure. In one or more embodiments, the system 200 may be representative of the computing system 100 illustrated in FIG. 1. The system 200 may be implemented using one or more instances of the computing system 100 described with reference to FIG. 1 or within other suitable computing environments as would be understood by one skilled in the art, such as those described with reference to FIGS. 10 and/or 11. In some embodiments, components of the system 200 may be implemented within a single computing environment that performs data processing, pathway-based analysis, and health-related recommendation generation in an integrated manner. In other embodiments, components of the system 200 may be distributed across multiple computing devices or networked environments configured to exchange biologically-related data sets, pathway-based representations, and analytical results in real-time. The components of the system 200 may be implemented using one or more software applications, specialized hardware accelerators, or combinations thereof configured to perform data integration, adaptive analytical processing, and iterative refinement operations associated with pathway-based health optimization. In one or more embodiments, the system 200 may operate within a continuous feedback loop in which newly received biologically-related data sets, updated pathway-based representations, and generated health-related recommendations are repeatedly processed to maintain an adaptive and current representation of patient health state, as described herein.

In one or more embodiments, the system 200 may be deployed across multiple healthcare, research, and operational contexts, including drug development, clinical trial execution, clinical management, and personal health monitoring. In drug development and clinical trial settings, the system 200 may support simulation of treatment response, cohort stratification, protocol evaluation, and adaptive study design through use of digital twins representing individual patients or patient populations. In clinical management environments, the system 200 may provide point-of-care decision support and patient-specific health-related recommendations, while in personal monitoring contexts, the system 200 may operate on biologically-related data sets provided by wearable devices, companion applications, or user input to support individualized health assessment and ongoing optimization of patient health states.

In some embodiments, the system 200 may generate and maintain one or more population-level or cohort-based virtual representations in addition to, or instead of, patient-specific virtual representations. Such population-level virtual representations may be constructed using aggregated, anonymized, or synthesized biologically-related data sets associated with multiple patients and may be used to model pathway behavior, response variability, or risk distribution across defined cohorts. The population-level virtual representations may support comparative analysis, extrapolation of patient-specific insights to broader populations, or evaluation of intervention strategies across groups, without requiring identification of individual patients.

The system 200 of FIG. 2 includes a multi-agent system 204, a data integration layer 206, a storage layer 208, a processing layer 210, a digital twin agentic system 212, and a data lake house 214. Together, these components define a layered and cooperative computational framework configured to receive, integrate, analyze, and transform biologically-related data sets into pathway-based representations of a patient to support generation of health-related recommendations. The multi-agent system 204 can coordinate data ingestion, task execution, and inter-component communication across the system 200. The data integration layer 206 processes and synchronizes the biologically-related data sets, while the storage layer 208 maintains integrated data and intermediate results generated during processing. The processing layer 210 performs analytical operations on the integrated data, and the digital twin agentic system 212 generates and refines a pathway-based virtual representation of the patient. The data lake house 214 aggregates and evaluates analytical results produced by the processing layer 210 and the digital twin agentic system 212 to produce an output 216, which may include health-related recommendations associated with the patient.

In one or more embodiments, the system 200 is configured to process a first input 202 and a second input 202' that provide biologically-related data sets to the components of the system 200. The first input 202 may include biologically-related data sets corresponding to one or more patients and received from external data sources, including clinical record systems, imaging repositories, molecular or multi-omics data sources, physiological monitoring devices, and other patient-associated data repositories. The second input 202′ may include biologically-related data sets generated within the system 200 or received subsequent to initial processing, including updated measurements, response data, intermediate analytical results, or feedback data associated with prior analyses. The first input 202 and the second input 202′ support iterative and ongoing processing by enabling the data integration layer 206, the processing layer 210, and the digital twin agentic system 212 to incorporate newly received or updated information when performing pathway-based analysis and generating health-related recommendations. However, it is understood that in some embodiments, the system 200 may execute the full sequence of data integration, pathway-based analysis, digital twin generation, and health-related recommendation generation using only the first input 202, without requiring the second input 202′.

In one or more embodiments, the multi-agent system 204 may be configured to coordinate and manage operations performed by the components of the system 200. The multi-agent system 204 may include one or more software agents configured to perform discrete functions associated with data ingestion, task orchestration, analytical execution, and result coordination across the data integration layer 206, the processing layer 210, the digital twin agentic system 212, and the data lake house 214. The multi-agent system 204 may control execution order, manage data dependencies, and facilitate communication among components to ensure that biologically-related data sets are processed in a consistent and orderly manner. In some embodiments, the multi-agent system 204 may enable parallel execution of tasks and dynamic routing of data or intermediate results to support scalable and efficient pathway-based health optimization.

In other embodiments, the multi-agent system comprises a distributed workflow wherein the one or more software agents are selected from: a user interaction agent, one or more patient data integration agents, one or more data source identification agents, one or more research agents, a cohort agent, one or more integration agents, a transformation agent, one or more validation agents, one or more analysis agents, one or more specialty agents, one or more data collection agents, one or more expert agents, one or more decision agents, one or more coordination agents, one or more visualization agents, or a combination thereof. Each software agent may operate independently within a function-specific scope, maintain synchronized communication with other agents, and contribute to one or more system-wide capabilities through coordinated actions.

In one or more embodiments, the data integration layer 206 may be configured to receive biologically-related data sets provided via the first input 202 and, when present, the second input 202′, and to integrate the biologically-related data sets into a unified data representation associated with a patient. The data integration layer 206 may perform operations including normalization, validation, temporal alignment, and synchronization across heterogeneous data types to ensure consistency and coherence of the biologically-related data sets. In some embodiments, the data integration layer 206 may identify and reconcile differences in data format, resolution, or update frequency among multiple data sources. The integrated data produced by the data integration layer 206 may be stored within the storage layer 208 and provided to the processing layer 210 and the digital twin agentic system 212 for subsequent pathway-based analysis.

In one or more embodiments, the storage layer 208 may be configured to store integrated biologically-related data sets and intermediate data generated during operation of the system 200. The storage layer 208 may maintain patient-associated data, including integrated data representations, historical records, intermediate analytical results, and outputs generated by the data integration layer 206, the processing layer 210, and the digital twin agentic system 212. In some embodiments, the storage layer 208 may include one or more data repositories or databases configured to organize and index stored information to support efficient retrieval and reuse during subsequent processing cycles. The storage layer 208 may further support persistence of data across iterative processing steps to enable ongoing analysis and refinement within the system 200.

In one or more embodiments, the processing layer 210 may be configured to perform analytical operations on integrated biologically-related data sets received from the data integration layer 206 or retrieved from the storage layer 208. The processing layer 210 may execute computational tasks associated with analyzing biological variation, identifying temporal or contextual patterns, and preparing data for pathway-based modeling. In some embodiments, the processing layer 210 may generate intermediate analytical results or derived features that are provided to the digital twin agentic system 212 for construction or refinement of a pathway-based virtual representation of a patient. The processing layer 210 may operate under the coordination of the multi-agent system 204 to ensure that analytical operations are executed in accordance with system state, data availability, and processing dependencies.

In one or more embodiments, the digital twin agentic system 212 may be configured to generate and refine a pathway-based virtual representation of a patient using integrated biologically-related data sets and analytical results produced by the processing layer 210. The digital twin agentic system 212 may construct a digital twin that models biological pathways, functional relationships, and biological state associated with the patient based on the integrated data. In some embodiments, the digital twin agentic system 212 may update the virtual representation as additional biologically-related data sets are received, including data provided via the second input 202′ or intermediate results generated during prior processing cycles. The pathway-based virtual representation generated by the digital twin agentic system 212 may be provided to the data lake house 214 for evaluation and use in generating health-related recommendations.

In one or more embodiments, the data lake house 214 may be configured to aggregate, evaluate, and manage analytical results generated by the processing layer 210 and the digital twin agentic system 212. The data lake house 214 may receive pathway-based representations, intermediate analytical outputs, and refined digital twin data associated with a patient, and may perform operations to assess consistency, relevance, and completeness of the received information. In some embodiments, the data lake house 214 may support evaluation of analytical results for purposes of generating health-related recommendations, conducting risk assessment, or supporting iterative refinement of pathway-based representations. The data lake house 214 may provide evaluated results to generate the output 216, which may include health-related recommendations associated with the patient for presentation to a user or downstream processing.

In other embodiments, the data lake house 214 is further configured to independently evaluate evidence and resolve one or more conflicts associated with the biologically-related data sets, including contradiction analysis, outcome integration, evidence weighting, or a combination thereof.

In one or more embodiments, the output 216 may include one or more health-related recommendations generated based on evaluation of pathway-based representations and analytical results within the data lake house 214. The output 216 may include recommendations associated with treatment, monitoring, intervention, or optimization of biologically-related solutions corresponding to a patient. In some embodiments, the output 216 may further include analytical summaries, visualization data, confidence indicators, or alerts derived from the pathway-based virtual representation of the patient. The output 216 may be provided to one or more users, including clinicians, patients, or authorized healthcare personnel, through user interfaces, dashboards, or connected systems to support interpretation and decision-making.

In some embodiments, the output 216 may additionally be transmitted to one or more external systems for automated or semi-automated downstream processing. Such external systems may include clinical decision-support platforms, monitoring applications, research analytics environments, or other computing systems configured to consume pathway-based representations or health-related recommendations. The transmitted output may include recommendation data, pathway-level indicators, confidence metrics, or updated digital twin state information, thereby enabling closed-loop integration between the system 200 and external computational environments without requiring direct therapeutic actuation or manual data re-entry.

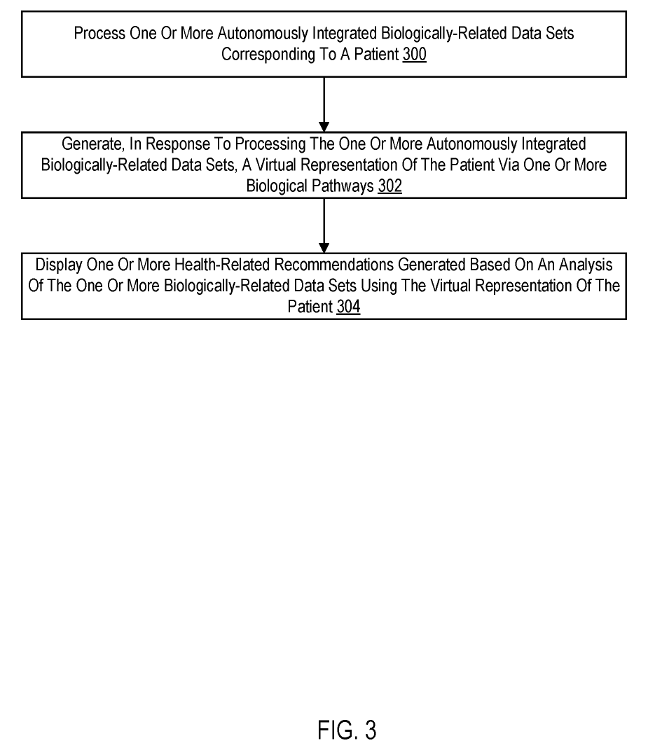

FIG. 2 therefore illustrates an example system architecture configured to perform the processes described in the subsequent figures. The multi-agent system 204, the data integration layer 206, the storage layer 208, the processing layer 210, the digital twin agentic system 212, and the data lake house 214 cooperate to receive and process biologically-related data sets, construct and refine a pathway-based digital twin of a patient, and generate health-related recommendations based on an analysis of the digital twin. The layered configuration of the system 200 supports iterative processing and incorporation of newly received or updated data, enabling continuous refinement of pathway-based representations and associated recommendations in real-time. In one or more embodiments, the system 200 may be implemented within local, distributed, or cloud-based computing environments that facilitate scalability, secure data exchange, and interoperability with external systems. FIG. 3 sets forth a flowchart illustrating an example method executed by the system 200, or the computing system 100, for pathway-based health optimization, including processing biologically-related data sets, generating a pathway-based virtual representation of a patient, and generating health-related recommendations in accordance with embodiments of the present disclosure.

For further explanation, FIG. 3 sets forth a flowchart illustrating an example method of pathway-based health optimization and generation of one or more health-related recommendations in accordance with embodiments of the present disclosure. The example method of FIG. 3 can be carried out in a system similar to that of FIG. 2. The method of FIG. 3 can be performed by the system 200 (which, in some embodiments, can be representative of the computing system 100), or by one or more components thereof, including a multi-agent system (e.g., the multi-agent system 204), a data integration layer (e.g., the data integration layer 206), a processing layer (e.g., the processing layer 210), a digital twin agentic system (e.g., the digital twin agentic system 212), and a data lake house (e.g., the data lake house 214).

The method of FIG. 3 includes processing 300 one or more autonomously integrated biologically-related data sets corresponding to a patient. Processing 300 the one or more autonomously integrated biologically-related data sets may be carried out by the data integration layer 206 in coordination with the multi-agent system 204. During processing 300 of the one or more autonomously integrated biologically-related data sets, the data integration layer 206 may autonomously obtain biologically-related data sets via the first input 202 and, when present, the second input 202′, without requiring manual intervention. The biologically-related data sets may include clinical records, imaging data, molecular or multi-omics data, physiological measurements, and other patient-associated data received from external systems or generated within the system 200.

In some embodiments, processing 300 the one or more autonomously integrated biologically-related data sets includes autonomously integrating the biologically-related data sets by the data integration layer 206 using rule-based workflows, agent-directed coordination, or automated data orchestration routines managed by the multi-agent system 204. The data integration layer 206 may aggregate the obtained biologically-related data sets and perform operations to normalize, validate, align, and synchronize the biologically-related data sets to produce integrated data suitable for downstream analysis. The data integration layer 206 may automatically normalize data formats, align temporal characteristics, reconcile measurement units, and synchronize update intervals across heterogeneous data sources. For example, when processing 300 the one or more autonomously integrated biologically-related data sets receives a combination of real-time physiological measurements and asynchronously updated molecular data, the data integration layer 206 may align the data sets to a common temporal reference and resolve inconsistencies in scale or resolution to maintain a unified integrated data representation.

In further embodiments, processing 300 the one or more autonomously integrated biologically-related data sets may also include autonomously monitoring the receipt of the biologically-related data sets and dynamically incorporating newly received or updated data into the integrated data representation as the data becomes available. The data integration layer 206 may detect changes in incoming data streams, identify data quality attributes, and selectively update portions of the integrated data representation without interrupting downstream analysis. For example, when updated physiological measurements or response data are received through the second input 202′, the data integration layer 206 may merge the updated information with previously integrated data to maintain a current and coherent representation of patient-associated biological information. Through processing 300 the one or more autonomously integrated biologically-related data sets, the data integration layer 206 produces integrated biologically-related data sets that serve as the foundation for the generation of a pathway-based virtual representation of the patient during subsequent method steps.

The method of FIG. 3 also includes generating 302 a virtual representation of the patient via one or more biological pathways. Generating 302 the virtual representation of the patient may be carried out by the digital twin agentic system 212 using the autonomously integrated biologically-related data sets produced during processing 300 the one or more autonomously integrated biologically-related data sets. During generating 302 the virtual representation of the patient, the digital twin agentic system 212 may analyze the integrated biologically-related data sets to identify biological pathways, pathway-level interactions, and functional relationships associated with the patient. The digital twin agentic system 212 may construct a pathway-based virtual representation of the patient that corresponds to a digital twin and models a biological state through computational representation of the identified pathways and relationships.

In some embodiments, generating 302 the virtual representation of the patient includes mapping elements of the integrated biologically-related data sets to corresponding biological pathways and associating pathway activity indicators with the mapped elements. The digital twin agentic system 212 may assign clinical measurements, molecular features, physiological signals, or response patterns to specific pathways and may represent interactions among the pathways within the virtual representation. For example, generating 302 the virtual representation of the patient may include associating longitudinal physiological measurements with pathway activity changes over time or incorporating molecular variation data to adjust pathway-level representations. Through generating 302 the virtual representation of the patient, the digital twin agentic system 212 produces a pathway-based virtual representation of the patient that provides a structured computational basis for downstream analysis and the generation of health-related recommendations.

The method of FIG. 3 also includes displaying 304 one or more health-related recommendations generated based on an analysis of the one or more biologically-related data sets using the virtual representation of the patient. Displaying 304 the one or more health-related recommendations may be carried out by the system 200, including the data lake house 214, and provided to a user as the output 216. During displaying 304 the one or more health-related recommendations, the data lake house 214 may evaluate the generated pathway-based virtual representation to derive one or more health-related recommendations associated with the patient. The output 216 may provide the one or more health-related recommendations for presentation to the user through a display screen as a GUI. For example, displaying 304 the one or more health-related recommendations may include presenting pathway-informed recommendation summaries, visualization data derived from the digital twin, or contextual indicators associated with the generated recommendations to clinicians, patients, or authorized healthcare personnel.

In some embodiments, displaying 304 the one or more health-related recommendations may include generating the one or more health-related recommendations based on an analysis performed using the pathway-based virtual representation of the patient prior to presentation to the user. The analysis may include evaluating pathway activity, biological variation, response patterns, or patient-specific characteristics represented within the digital twin. For example, the data lake house 214 may analyze interactions among biological pathways, temporal changes reflected in the virtual representation, or associations between integrated biologically-related data sets and observed patient responses to identify candidate recommendations. In such embodiments, the generated health-related recommendations may be selected, ranked, or contextualized based on pathway-level relationships represented within the virtual representation before being provided for display to the user.

In other embodiments, the health-related recommendations generated during displaying 304 the one or more health-related recommendations may be accompanied by explanatory metadata that identifies contributing biological pathways, causal factors, or analytical inputs that influenced the generation of the recommendation. Such explanatory metadata may include pathway attribution scores, confidence indicators, causal dependency relationships, or provenance information derived from the digital twin and associated analytical processes. The explanatory metadata may support auditability, interpretability, and expert review of the recommendations by enabling a user or external system to trace the recommendation to underlying pathway-level contributors without requiring disclosure of raw data or model internals.

The method steps of FIG. 3 collectively describe a process for pathway-based health optimization using integrated biologically-related data sets. Processing 300 the one or more autonomously integrated biologically-related data sets, generating 302 the virtual representation of the patient, and displaying 304 the one or more health-related recommendations cooperate to enable the system 200 to autonomously integrate biologically-related data, construct a pathway-based digital twin of a patient, and generate and present health-related recommendations based on an analysis of the digital twin. In one or more embodiments, the method of FIG. 3 may be executed repeatedly or continuously to incorporate newly received or updated biologically-related data sets, thereby supporting ongoing refinement of the virtual representation and corresponding health-related recommendations over time.

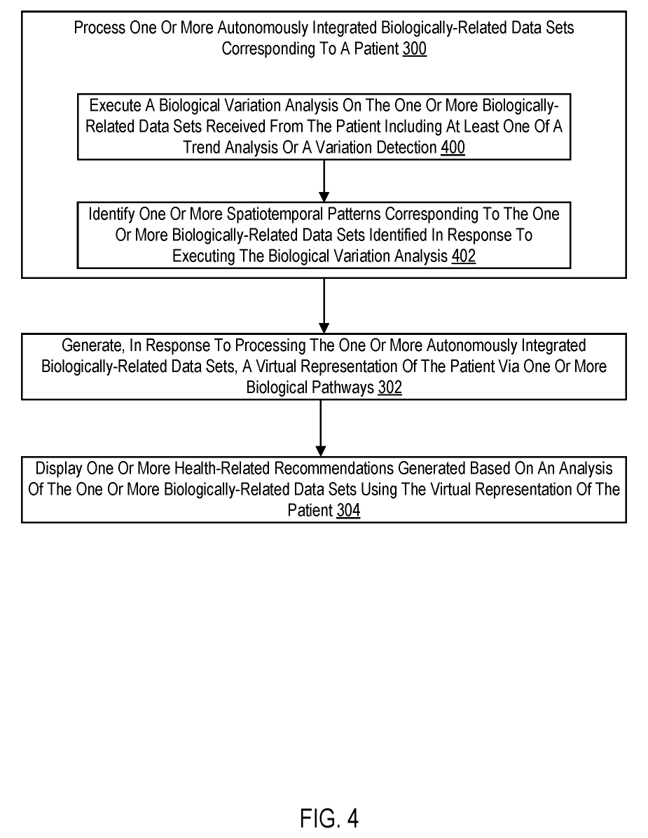

FIG. 3 therefore illustrates an example method that defines the operational flow executed by the system 200 for pathway-based health optimization. The sequence of steps shown in FIG. 3 demonstrates how the system 200 processes autonomously integrated biologically-related data sets, generates a pathway-based virtual representation of a patient, and analyzes the virtual representation to generate and display health-related recommendations. The flowchart of FIG. 3 provides a foundation for the subsequent figures, which further expand upon the operations shown. FIG. 4, in particular, sets forth a flowchart illustrating an example method that expands upon the operations described with reference to FIG. 3 by detailing the manner in which the system 200 performs biological variation analysis and identifies spatiotemporal patterns associated with the biologically-related data sets in accordance with embodiments of the present disclosure.

For further explanation, FIG. 4 sets forth a flowchart illustrating an example method of pathway-based health optimization that expands upon the operations described with reference to FIG. 3 in accordance with embodiments of the present disclosure. The example method of FIG. 4 can be carried out in a system similar to that of FIG. 2. The method of FIG. 4 can be performed by the system 200 (which, in some embodiments, can be representative of the computing system 100) or by one or more components thereof, including a multi-agent system (e.g., the multi-agent system 204), a data integration layer (e.g., the data integration layer 206), a processing layer 210 (e.g., the processing layer 210), a digital twin agentic system 212 (e.g., the digital twin agentic system 212), and a data lake house (e.g., the data lake house 214).

The method of FIG. 4 includes executing 400 a biological variation analysis on the one or more biologically-related data sets received from the patient as part of processing 300 the one or more autonomously integrated biologically-related data sets described with reference to FIG. 3. Executing 400 the biological variation analysis may be carried out by the processing layer 210 in coordination with the data integration layer 206. During executing 400 the biological variation analysis, the processing layer 210 may analyze the integrated biologically-related data sets to identify a variation in biological measurements over time, including changes in physiological signals, molecular profiles, imaging-derived parameters, or other patient-associated biological indicators. The biological variation analysis may include, for example, performing trend analysis to detect directional changes in measured values or variation detection to identify deviations from baseline or expected biological ranges, as described herein. However, it is understood that the biological variation analysis may also include other analyses or means for detection used for health-related purposes.

In some embodiments, executing 400 the biological variation analysis may include analyzing a biological variation across multiple dimensions represented within the integrated biologically-related data sets. The processing layer 210 may evaluate temporal trends, rate-of-change characteristics, or variability metrics associated with physiological measurements, molecular markers, or pathway-associated features represented within the data. For example, executing 400 the biological variation analysis may include comparing current measurements to historical baselines, identifying periodic or cyclical patterns, or detecting abrupt changes indicative of biological response or external influence. In such embodiments, results of the biological variation analysis may be stored within the storage layer 208 or provided to the digital twin agentic system 212 to inform subsequent identification of spatiotemporal patterns and refinement of the pathway-based virtual representation of the patient.

The method of FIG. 4 also includes identifying 402 one or more spatiotemporal patterns corresponding to the one or more biologically-related data sets, identified in response to executing 400 the biological variation analysis. As is depicted in FIG. 4, identifying 402 the one or more spatiotemporal patterns is also performed as part of processing 300 the one or more autonomously integrated biologically-related data sets described with reference to FIG. 3. Identifying 402 the one or more spatiotemporal patterns may be carried out by the processing layer 210 in coordination with the digital twin agentic system 212. During identifying 402 the one or more spatiotemporal patterns, the processing layer 210 may analyze variation results produced during executing 400 the biological variation analysis to determine relationships between biological measurements and temporal or contextual factors, including timing, duration, frequency, or co-occurrence of observed biological changes. For example, identifying 402 the one or more spatiotemporal patterns may include correlating detected trends or variations with temporal sequences, physiological cycles, or pathway-level interactions represented within the integrated biologically-related data sets. The identified spatiotemporal patterns may be provided to the digital twin agentic system 212 for incorporation into the pathway-based virtual representation of the patient, as described herein.

In some embodiments, identifying 402 the one or more spatiotemporal patterns may include detecting spatiotemporal patterns that reflect interactions between biological variation and patient-specific context represented within the integrated biologically-related data sets. The processing layer 210 may evaluate associations between variation results and spatial attributes, temporal intervals, or pathway-specific activity states to identify recurring, progressive, or transient patterns. For example, identifying 402 the one or more spatiotemporal patterns may include determining that changes in a physiological measurement occur consistently within defined temporal windows, coincide with activation or suppression of particular biological pathways, or correlate with external conditions reflected in the biologically-related data sets. In such embodiments, the identified spatiotemporal patterns may be stored within the storage layer 208 or provided to the digital twin agentic system 212 to support refinement of the pathway-based virtual representation and downstream generation of health-related recommendations.

In further embodiments, executing 400 the biological variation analysis and identifying 402 the one or more spatiotemporal patterns may further include generating one or more genetic or molecular impact scores that quantify relative contributions of genetic variants, molecular features, or expression patterns to pathway-level biological behavior. Such impact scores may be used to weight pathway activity, adjust pathway interactions, or prioritize biological signals within the virtual representation. The impact scores may be generated using statistical, machine-learning, or rules-based techniques and may be incorporated into the pathway-based virtual representation without requiring a fixed scoring scale or predefined threshold.

The method steps of FIG. 4 collectively describe operations performed by the system 200 for analyzing biological variation and identifying spatiotemporal patterns within integrated biologically-related data sets. Generating 302 the virtual representation of the patient and displaying 304 the one or more health-related recommendations may each be performed as described with reference to FIG. 3, while executing 400 the biological variation analysis and identifying 402 the one or more spatiotemporal patterns expand upon processing 300 the one or more autonomously integrated biologically-related data sets representing analytical operations in which the system 200 evaluates variation in biological measurements and determines temporal and contextual patterns associated with the biologically-related data sets. The operations of FIG. 4 enable the system 200 to incorporate temporal and pathway-level biological behavior into a pathway-based virtual representation of a patient prior to generating and displaying health-related recommendations.

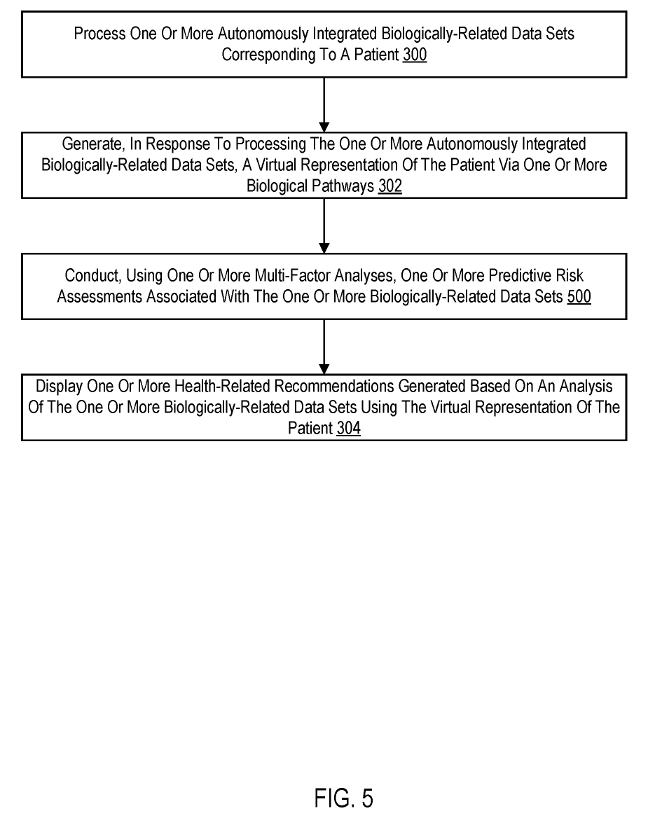

FIG. 4 therefore illustrates an example method executed by the system 200 for analyzing biological variation and identifying spatiotemporal patterns within integrated biologically-related data sets to enhance pathway-based health optimization. The sequence of operations shown in FIG. 4 demonstrates how the system 200 incorporates temporal and pathway-level biological behavior into a pathway-based virtual representation of a patient prior to generation and display of health-related recommendations. FIG. 5 sets forth a flowchart illustrating an example method that expands upon the operations described with reference to FIG. 3 by detailing the manner in which the system 200 conducts predictive risk assessment using the biologically-related data sets in accordance with embodiments of the present disclosure.

For further explanation, FIG. 5 sets forth a flowchart illustrating an example method of conducting predictive risk assessment using one or more biologically-related data sets in accordance with embodiments of the present disclosure. The example method of FIG. 5 can be carried out in a system similar to that of FIG. 2. The method of FIG. 5 can be performed by the system 200 (which, in some embodiments, can be representative of the computing system 100) or by one or more components thereof, including a processing layer (e.g., the processing layer 210), a digital twin agentic system (e.g., the digital twin agentic system 212), and a data lake house (e.g., the data lake house 214).

The method of FIG. 5 includes conducting 500 one or more predictive risk assessments associated with the one or more biologically-related data sets. Conducting 500 the one or more predictive risk assessments may be carried out by the data lake house 214 in coordination with the processing layer 210 and the digital twin agentic system 212. During conducting 500 the one or more predictive risk assessments, the data lake house 214 may analyze the generated 302 pathway-based virtual representation of the patient together with the integrated biologically-related data sets produced during processing 300 the one or more autonomously integrated biologically-related data sets. The predictive risk assessment may include evaluating patient-specific risk indicators associated with biological pathways, variation patterns, or temporal trends represented within the digital twin. For example, conducting 500 the one or more predictive risk assessments may include assessing likelihoods of adverse outcomes, progression of biological conditions, or response-related risks based on pathway-level relationships and interactions captured within the virtual representation.

In some embodiments, conducting 500 the one or more predictive risk assessments includes generating one or more predictive outputs that quantify future risk associated with the patient based on analysis of the pathway-based virtual representation. The data lake house 214 may apply predictive models or risk estimation techniques to forecast likelihoods of future biological events, adverse outcomes, or progression of patient-specific conditions over one or more future time intervals. For example, conducting 500 the one or more predictive risk assessments may include predicting the probability of pathway dysregulation, anticipated response to an intervention, or projected deviation from a baseline biological state based on current pathway activity and identified variation patterns. In such embodiments, the predictive outputs generated during conducting 500 the one or more predictive risk assessments may include risk scores, probability estimates, or categorical risk classifications that are subsequently used to inform generation and display 304 of the one or more health-related recommendations.

In some embodiments, the conducted one or more predictive risk assessments may incorporate causal inference results or variant impact scores derived from prior analysis steps to improve attribution of risk to specific biological pathways or mechanisms. By integrating causal relationships or pathway-level contribution measures, the predictive risk assessment may distinguish modifiable biological drivers from correlated indicators, thereby supporting generation of health-related recommendations that reflect underlying biological causality rather than surface-level statistical association.

In other embodiments, the autonomous digital twin agentic system 212 is further configured to independently initiate self-directed research associated with the biologically-related data sets, autonomously generate one or more hypotheses based on emerging biological patterns, test the generated hypotheses, and validate evidence derived from real-world data, multi-media research sources, or a combination thereof.

The method steps of FIG. 5 collectively describe operations performed by the system 200 for conducting 500 the one or more predictive risk assessments using integrated biologically-related data sets and a pathway-based virtual representation of a patient. Processing 300 the one or more autonomously integrated biologically-related data sets and generating 302 the virtual representation of the patient may each be performed as described with reference to FIG. 3, while conducting 500 the one or more predictive risk assessments represents an intermediate analytical operation in which the data lake house 214 evaluates pathway-level relationships, biological variation, and identified spatiotemporal patterns to generate forward-looking risk predictions. The operations of FIG. 5 enable the system 200 to assess potential future biological outcomes, patient-specific conditions, or response-related risks prior to the presentation of health-related recommendations.

In one or more embodiments, displaying 304 the one or more health-related recommendations is also performed as described with reference to FIG. 3. In such embodiments, displaying 304 the one or more health-related recommendations may be performed after conducting 500 the one or more predictive risk assessments, such that the one or more health-related recommendations presented to the user are generated based on the analysis of the virtual representation of the patient as further informed by the predictive risk assessment results. Accordingly, FIG. 5 illustrates that conducting 500 the one or more predictive risk assessments is performed prior to displaying 304 the one or more health-related recommendations, while the manner of displaying remains consistent with displaying 304 the one or more health-related recommendations described with reference to FIG. 3.

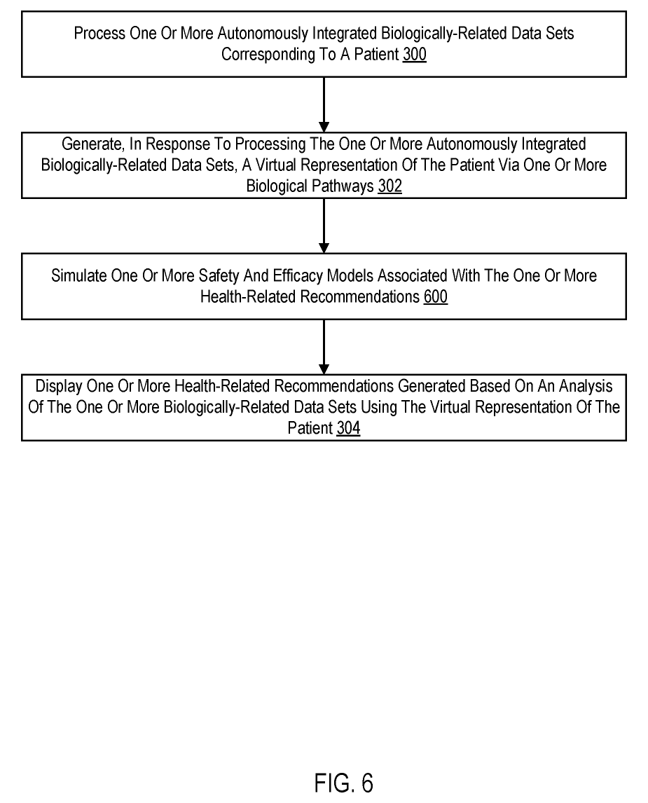

FIG. 5 therefore illustrates an example method executed by the system 200 for incorporating predictive risk assessment into pathway-based health optimization. The sequence of operations shown in FIG. 5 demonstrates how the system 200 transitions from generation of a pathway-based virtual representation of a patient to evaluation of future risk using predictive analysis. FIG. 6 sets forth a flowchart illustrating an example method that expands upon the operations described with reference to FIG. 3 by detailing the manner in which the system 200 simulates one or more safety and efficacy models associated with the health-related recommendations in accordance with embodiments of the present disclosure.

For further explanation, FIG. 6 sets forth a flowchart illustrating an example method of simulating one or more safety and efficacy models associated with health-related recommendations in accordance with embodiments of the present disclosure. The example method of FIG. 6 can be carried out in a system similar to that of FIG. 2. The method of FIG. 6 can be performed by the system 200 (which, in some embodiments, may be representative of the computing system 100) or by one or more components thereof, including a processing layer (e.g., the processing layer 210), a digital twin agentic system (e.g., the digital twin agentic system 212), and a data lake house (e.g., the data lake house 214).

The method of FIG. 6 includes simulating 600 one or more safety and efficacy models associated with the one or more health-related recommendations. Simulating 600 the one or more safety and efficacy models may be carried out by the data lake house 214 in coordination with the digital twin agentic system 212. During simulating 600 the one or more safety and efficacy models, the data lake house 214 may evaluate the generated 302 pathway-based virtual representation of the patient together with analytical results produced during processing 300 the one or more autonomously integrated biologically-related data sets. The safety and efficacy simulation may include assessing potential outcomes, constraints, or response characteristics associated with one or more candidate health-related recommendations based on pathway-level relationships represented within the digital twin.

In some embodiments, simulating 600 the one or more safety and efficacy models includes generating modeled outcomes that estimate safety margins, efficacy likelihoods, or potential adverse effects associated with the one or more health-related recommendations over one or more simulated conditions or time intervals. The data lake house 214 may apply simulation techniques that evaluate interactions among biological pathways, predicted risk factors identified during conducting 500 the one or more predictive risk assessments, and patient-specific characteristics represented within the pathway-based virtual representation. For example, simulating 600 the one or more safety and efficacy models may include estimating whether a health-related recommendation remains within predefined safety thresholds, achieves a desired biological effect, or introduces elevated risk based on projected pathway responses. In such embodiments, results of the safety and efficacy simulation may be stored within the storage layer 208 or used to qualify, rank, or filter health-related recommendations prior to presentation during displaying 304 the one or more health-related recommendations.

In further embodiments, simulating 600 the one or more safety and efficacy models may be performed using advanced computational techniques configured to model biological interactions at varying levels of resolution. Such techniques may include classical simulation methods, quantum-enhanced optimization techniques, or hybrid computational approaches that evaluate molecular interactions, pathway responses, or system-level biological behavior. The simulation techniques may be selected based on available computing resources, desired precision, or complexity of the biological interactions being modeled, without requiring the use of any specific computational paradigm.

The method steps of FIG. 6 collectively describe operations performed by the system 200 for simulating 600 the safety and efficacy of one or more health-related recommendations using integrated biologically-related data sets and a pathway-based virtual representation of a patient. Processing 300 the one or more autonomously integrated biologically-related data sets, generating 302 the virtual representation of the patient, and conducting 500 the one or more predictive risk assessments may each be performed as described with reference to FIGS. 3 and 5, while simulating 600 the one or more safety and efficacy models represents an evaluative analytical operation in which the data lake house 214 assesses projected biological responses, safety constraints, and efficacy characteristics associated with candidate health-related recommendations. The operations of FIG. 6 enable the system 200 to evaluate anticipated outcomes and potential risks associated with the health-related recommendations prior to, or in conjunction with, presentation to a user.

In one or more embodiments, displaying 304 the one or more health-related recommendations is also performed as described with reference to FIG. 3. In such embodiments, displaying 304 the one or more health-related recommendations may be performed after simulating 600 the one or more safety and efficacy models, such that the one or more health-related recommendations provided to the user reflect modeled safety constraints, efficacy projections, or simulated outcome characteristics derived from the virtual representation of the patient. Thus, FIG. 6 illustrates that simulating 600 the one or more safety and efficacy models precedes displaying 304 the one or more health-related recommendations, while the display operation itself corresponds to displaying 304 the one or more health-related recommendations described with reference to FIG. 3.

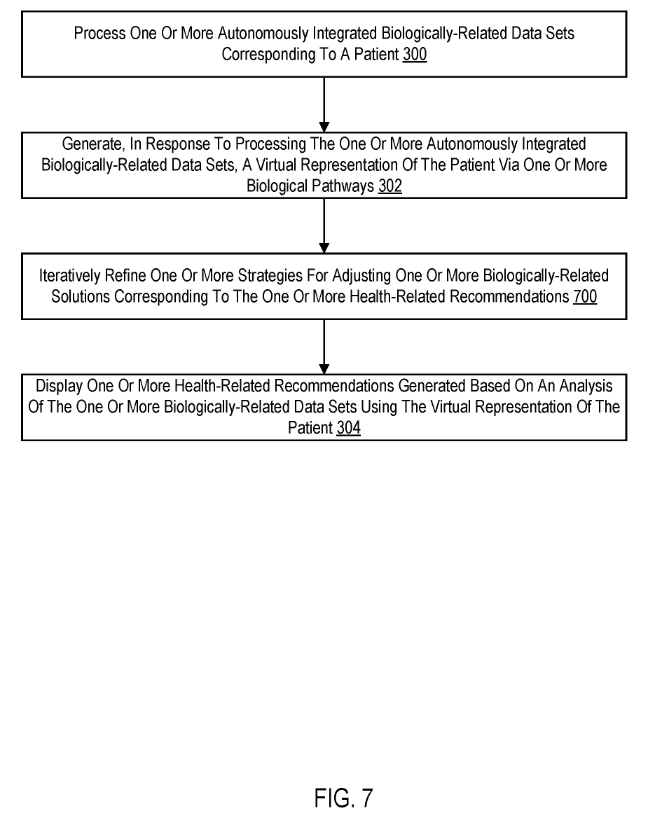

FIG. 6 therefore illustrates an example method executed by the system 200 for incorporating safety and efficacy simulation into pathway-based health optimization. The sequence of operations shown in FIG. 6 demonstrates how the system 200 transitions from predictive risk assessment to evaluation of modeled safety and efficacy using a pathway-based virtual representation of a patient. FIG. 7 sets forth a flowchart illustrating an example method that expands upon the operations described with reference to FIG. 3 by detailing the manner in which the system 200 iteratively refines one or more strategies for adjusting biologically-related solutions corresponding to the health-related recommendations in accordance with embodiments of the present disclosure.

For further explanation, FIG. 7 sets forth a flowchart illustrating an example method of iteratively refining one or more strategies for adjusting biologically-related solutions corresponding to health-related recommendations in accordance with embodiments of the present disclosure. The example method of FIG. 7 can be carried out in a system similar to that of FIG. 2. The method of FIG. 7 can be performed by the system 200 (which, in some embodiments, can be representative of the computing system 100) or by one or more components thereof, including a processing layer (e.g., the processing layer 210), a digital twin agentic system (e.g., the digital twin agentic system 212), and a data lake house (e.g., the data lake house 214).

The method of FIG. 7 includes iteratively refining 700 one or more strategies for adjusting one or more biologically-related solutions corresponding to the one or more health-related recommendations. Iteratively refining 700 the one or more strategies may be carried out by the data lake house 214 in coordination with the digital twin agentic system 212. During iteratively refining 700 the one or more strategies, the system 200 may first identify one or more candidate strategies from stored strategy sets, protocol repositories, or previously generated recommendation records maintained within a storage layer (e.g., the storage layer 208) or the data lake house 214. The identified strategies may be selected based on an analysis of the generated 302 pathway-based virtual representation of the patient together with analytical results produced during processing 300 the one or more autonomously integrated biologically-related data sets. The data lake house 214 may then iteratively evaluate and adjust the identified strategies by modifying parameters, selection criteria, or prioritization associated with the biologically-related solutions to improve alignment with a patient-specific biological state, predicted risk, and modeled safety and efficacy characteristics represented within the pathway-based virtual representation.

In some embodiments, iteratively refining 700 the one or more strategies includes updating the identified strategies based on feedback derived from predicted outcomes, simulated responses, or changes reflected in newly received biologically-related data sets. The data lake house 214 may re-evaluate pathway-level interactions, predictive risk indicators generated during conducting 500 the one or more predictive risk assessments, and safety and efficacy results generated during simulating 600 the one or more safety and efficacy models as additional information becomes available. For example, iteratively refining 700 the one or more strategies may include modifying adjustment parameters, reordering candidate strategies, or selectively excluding strategies that no longer satisfy patient-specific biological constraints represented within the pathway-based virtual representation. In such embodiments, the iterative refinement supports continuous adaptation of the health-related recommendations in response to evolving patient conditions.

In further embodiments, iteratively refining 700 the one or more strategies may be performed using distributed or federated computational techniques in which portions of the biologically-related data sets or intermediate analytical results remain localized to separate computing nodes. The system 200 may coordinate refinement of strategies across multiple nodes while preserving data locality, privacy constraints, or regulatory requirements. Such distributed refinement techniques may support scalability, privacy-preserving analysis, or deployment across edge devices, cloud environments, or hybrid architectures.

The method steps of FIG. 7 collectively describe operations performed by the system 200 for iteratively refining 700 the one or more strategies for adjusting biologically-related solutions corresponding to health-related recommendations. Processing 300 the one or more autonomously integrated biologically-related data sets, generating 302 the virtual representation of the patient, conducting 500 the one or more predictive risk assessments, and simulating 600 the one or more safety and efficacy models may each be performed as described with reference to FIGS. 3, 5, and 6, while iteratively refining 700 the one or more strategies represents an adaptive operation in which the system 200 identifies candidate strategies and incrementally adjusts those strategies based on patient-specific biological state, predicted risk, and modeled safety and efficacy outcomes. The operations of FIG. 7 enable the system 200 to improve alignment between the health-related recommendations and evolving biological conditions associated with the patient prior to presentation.

In one or more embodiments, displaying 304 the one or more health-related recommendations is also performed as described with reference to FIG. 3. In such embodiments, displaying 304 the one or more health-related recommendations may be performed after iteratively refining 700 the one or more strategies for adjusting biologically-related solutions, such that the one or more health-related recommendations presented to the user reflect the refined strategies informed by the virtual representation of the patient. Accordingly, FIG. 7 illustrates that iteratively refining 700 the one or more strategies is performed prior to displaying 304 the one or more health-related recommendations, while the displaying operation remains consistent with displaying 304 the one or more health-related recommendations described with reference to FIG. 3.

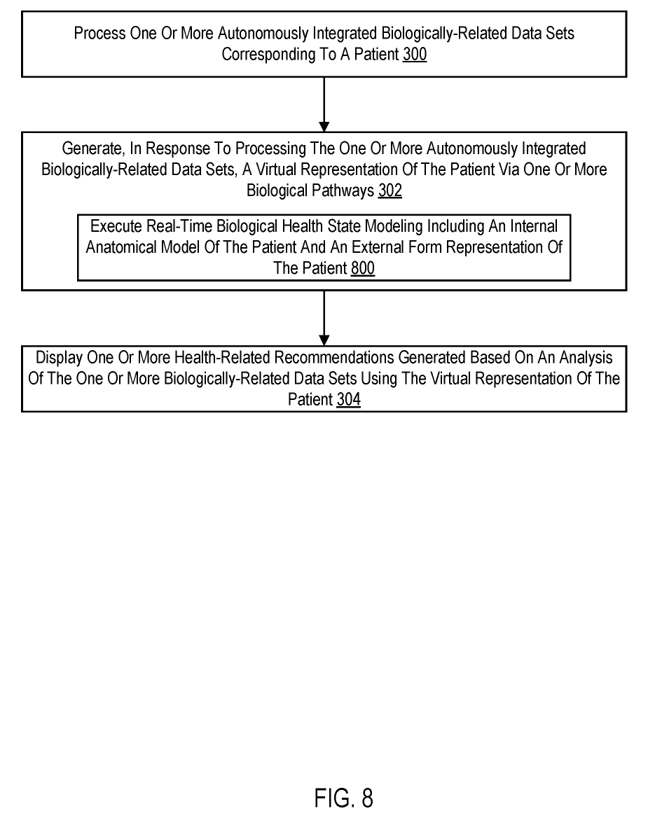

FIG. 7 therefore illustrates an example method executed by the system 200 for adaptively refining strategies associated with health-related recommendations in response to patient-specific biological conditions. The sequence of operations shown in FIG. 7 demonstrates how the system 200 transitions from generation, risk assessment, and simulation of candidate recommendations to iterative adjustment of biologically-related solutions using a pathway-based virtual representation of a patient. FIG. 8 sets forth a flowchart illustrating an example method that expands upon the operations described with reference to FIG. 3 by detailing the manner in which the system 200 performs real-time biological health state modeling, including generation of an internal anatomical model and an external form representation of the patient, in accordance with embodiments of the present disclosure.

For further explanation, FIG. 8 sets forth a flowchart illustrating an example method of executing real-time biological health state modeling in accordance with embodiments of the present disclosure. The example method of FIG. 8 can be carried out in a system similar to that of FIG. 2. The method of FIG. 8 can be performed by the system 200 (which, in some embodiments, may be representative of the computing system 100) or by one or more components thereof, including a processing layer (e.g., the processing layer 210), a digital twin agentic system (e.g., the digital twin agentic system 212), and a data lake house (e.g., the data lake house 214).

The method of FIG. 8 includes executing 800 real-time biological health state modeling, including an internal anatomical model of the patient and an external form representation of the patient, as part of generating 302 the virtual representation of the patient described with reference to FIG. 3. Executing 800 the real-time biological health state modeling may be carried out by the digital twin agentic system 212 in coordination with the processing layer 210. During executing 800 the real-time biological health state modeling, the digital twin agentic system 212 may use the autonomously integrated biologically-related data sets produced during processing 300 the one or more autonomously integrated biologically-related data sets to construct and update anatomical and form-based representations associated with the patient. The real-time biological health state modeling may include, for example, updating internal anatomical structures based on imaging or physiological data, adjusting external form characteristics based on observed biological measurements, or synchronizing anatomical and form representations with temporal changes reflected in newly received biologically-related data sets, as described herein. However, it is understood that the real-time biological health state modeling may also include other modeling techniques or representational approaches used to reflect patient health state for health-related purposes.

In some embodiments, executing 800 the real-time biological health state modeling includes continuously updating the internal anatomical model and the external form representation in response to newly received or updated biologically-related data sets provided during processing 300 the one or more autonomously integrated biologically-related data sets. The digital twin agentic system 212 may incorporate real-time imaging data, physiological measurements, or other patient-associated biological inputs to dynamically adjust anatomical geometry, spatial relationships, or form characteristics represented within the pathway-based virtual representation. For example, executing 800 the real-time biological health state modeling may include updating organ dimensions, tissue characteristics, or external form features to reflect temporal changes observed in the biologically-related data sets. In such embodiments, the real-time biological health state modeling supports maintenance of a current and synchronized representation of patient health state that may be used during subsequent analysis and generation of health-related recommendations.

The method steps of FIG. 8 collectively describe operations performed by the system 200 for executing 800 the real-time biological health state modeling using integrated biologically-related data sets. Processing 300 the one or more autonomously integrated biologically-related data sets and displaying 304 the one or more health-related recommendations may each be performed as described with reference to FIG. 3, while executing 800 the real-time biological health state modeling expands upon generating 302 the virtual representation of the patient to represent a modeling operation in which the system 200 constructs and updates an internal anatomical model and an external form representation of a patient. The operations of FIG. 8 enable the system 200 to maintain a synchronized representation of patient health state that reflects anatomical structure and external form characteristics for use in downstream analysis and presentation of health-related recommendations.

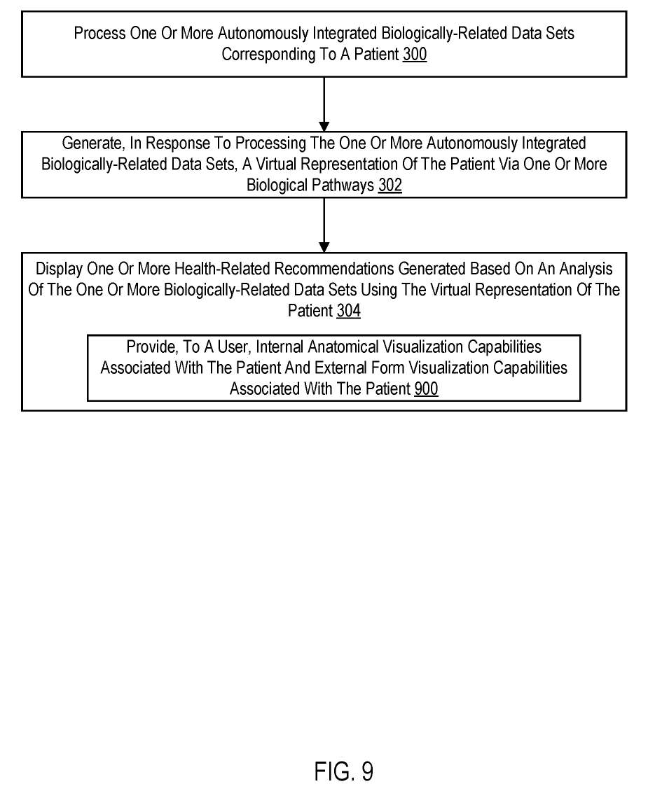

FIG. 8 therefore illustrates an example method executed by the system 200 for incorporating real-time biological health state modeling into pathway-based health optimization. The sequence of operations shown in FIG. 8 demonstrates how the system 200 augments a pathway-based virtual representation of a patient with internal anatomical modeling and external form representation derived from integrated biologically-related data sets. FIG. 9 sets forth a flowchart illustrating an example method that expands upon the operations described with reference to FIG. 3 by detailing the manner in which the system 200 provides internal anatomical visualization capabilities and external form visualization capabilities to a user in accordance with embodiments of the present disclosure.

For further explanation, FIG. 9 sets forth a flowchart illustrating an example method of providing internal anatomical visualization capabilities and external form visualization capabilities to a user in accordance with embodiments of the present disclosure. The example method of FIG. 9 can be carried out in a system similar to that of FIG. 2. The method of FIG. 9 can be performed by the system 200 (which, in some embodiments, may be representative of the computing system 100) or by one or more components thereof, including a digital twin agentic system (e.g., the digital twin agentic system 212) and a data lake house (e.g., the data lake house 214).

The method of FIG. 9 includes providing 900 internal anatomical visualization capabilities and external form visualization capabilities to a user as part of displaying 304 the one or more health-related recommendations described with reference to FIG. 3. Providing 900 the internal anatomical visualization capabilities and the external form visualization capabilities may be carried out by the data lake house 214 in coordination with the digital twin agentic system 212. During providing 900 the internal anatomical visualization capabilities and the external form visualization capabilities, the data lake house 214 may use the autonomously integrated biologically-related data sets produced during processing 300 the one or more autonomously integrated biologically-related data sets together with the generated 302 pathway-based virtual representation of the patient to produce visualization outputs. The internal anatomical visualization capabilities may include, for example, graphical representations of anatomical structures, spatial relationships, or pathway-associated features derived from the internal anatomical model of the patient, while the external form visualization capabilities may include graphical representations of external form characteristics or form-based changes reflected in the external form representation of the patient. However, it is understood that providing 900 the internal anatomical visualization capabilities and the external form visualization capabilities may also include other visualization techniques or presentation formats used for health-related purposes.

In some embodiments, providing 900 the internal anatomical visualization capabilities and the external form visualization capabilities includes updating the internal anatomical visualization capabilities and the external form visualization capabilities in real-time as newly received biologically-related data sets are integrated during processing 300 the one or more autonomously integrated biologically-related data sets. The digital twin agentic system 212 may adjust visualization parameters, rendering detail, or presentation views based on changes reflected in the pathway-based virtual representation of the patient. For example, providing 900 the internal anatomical visualization capabilities and the external form visualization capabilities may include dynamically highlighting anatomical regions associated with altered pathway activity, updating external form representations to reflect observed physiological changes, or synchronizing visualization outputs with updated health-related recommendations. In such embodiments, the visualization capabilities support continuous interpretation of a health state of a patient and provide contextual insight into the biological basis of the generated health-related recommendations.

The method steps of FIG. 9 collectively describe operations performed by the system 200 for providing 900 the internal anatomical visualization capabilities and the external form visualization capabilities to the user based on integrated biologically-related data sets. Processing 300 the one or more autonomously integrated biologically-related data sets and generating 302 the virtual representation of the patient may each be performed as described with reference to FIG. 3, while providing 900 the internal anatomical visualization capabilities and the external form visualization capabilities expands upon displaying 304 the one or more health-related recommendations to represent a presentation operation in which the system 200 renders visual outputs derived from a pathway-based virtual representation of a patient. The operations of FIG. 9 enable the system 200 to present anatomically and form-informed visualizations that support interpretation of a health state of a patient and associated health-related recommendations.

FIG. 9 therefore illustrates an example method executed by the system 200 for presenting internal anatomical visualization capabilities and external form visualization capabilities derived from a pathway-based virtual representation of a patient. The sequence of operations shown in FIG. 9 demonstrates how the system 200 translates integrated biologically-related data sets and real-time biological health state modeling into visual outputs that support user understanding of a health state of a patient and associated health-related recommendations. FIG. 10 sets forth a block diagram illustrating an example computing environment in which the system 200 and the methods described with reference to FIGS. 3-9 may be implemented, including cloud-based, distributed, or hybrid architectures configured to support scalable processing, data management, and execution of pathway-based health optimization operations in accordance with embodiments of the present disclosure.

For further explanation, FIG. 10 sets forth a block diagram of a cloud computing environment suitable for implementing one or more embodiments of the present disclosure. The cloud computing environment of FIG. 10 may be used to deploy and manage the system 200 and the computing system 100 for pathway-based health optimization. As shown in FIG. 10, a cloud service provider 1002 may deliver computing, platform, and software resources through a service-based consumption model in which resources are provisioned on demand and accessed as managed services. One or more clients 1032 may access the cloud service provider 1002 through a network 1034, which may include the Internet, a private healthcare network, or a hybrid infrastructure configured to support secure and efficient data exchange between distributed systems, healthcare providers, research environments, and end-user devices. The cloud service provider 1002 may operate within a public, private, or hybrid cloud configuration to support scalability, reliability, and interoperability across distributed components of the environment.

FIG. 10 depicts an embodiment in which software 1020 is delivered as a service. Software-as-a-Service (SaaS) provides access to software applications hosted by the cloud service provider 1002 over the network 1034 without requiring local installation or maintenance within clinical, research, or operational environments. As examples of the software 1020 delivered as a service, the illustrated embodiment includes office productivity 1022 software, customer relationship management (CRM) 1024 software, and project management 1026 software. In the context of pathway-based health optimization, additional software services may include applications for managing biologically-related data sets, presenting pathway-based virtual representations of patients, and displaying health-related recommendations generated by the system 200 and the computing system 100.

FIG. 10 also depicts an embodiment in which platform 1012 resources are delivered as a service. Platform-as-a-Service (PaaS) provides managed environments that enable developers, healthcare organizations, and research teams to build, deploy, and scale applications for pathway-based health optimization without maintaining underlying infrastructure. As examples of the platform 1012 resources, the illustrated embodiment includes database 1014 services, development tools 1016 services, and execution runtime 1018 services. The database 1014 services may provide scalable and secure storage for biologically-related data sets, pathway-based virtual representations, digital twin records, and analytical results generated by the system 200. The development tools 1016 services may support development, testing, and deployment of applications that interface with the system 200 and the computing system 100, while the execution runtime 1018 services may provide managed computing environments capable of executing data integration, analytical processing, simulation, and iterative refinement workloads associated with pathway-based health optimization.

FIG. 10 further depicts an embodiment in which infrastructure 1004 resources are delivered as a service. Infrastructure-as-a-Service (IaaS) provides virtualized computing hardware resources that include compute 1006, storage 1008, and networking 1010 capabilities. The compute 1006 resources may include virtual machines, containers, or specialized processing resources configured to execute data integration, pathway-based analysis, simulation, and predictive modeling operations performed by the system 200 and the computing system 100. The storage 1008 resources may include scalable block storage or object storage configured to securely maintain biologically-related data sets, pathway-based virtual representations, digital twin records, and intermediate analytical results. The networking 1010 resources may include virtual networks, secure gateways, or private connectivity services that enable reliable and encrypted communication between distributed system components, external data sources, and client devices.

FIG. 10 further depicts an embodiment in which the cloud service provider 1002 delivers security 1028 and management 1030 resources as part of the cloud-based environment. The security 1028 resources may include encryption services, identity and access management, authentication mechanisms, and monitoring capabilities configured to protect biologically-related data sets, pathway-based virtual representations, and communications associated with the system 200 and the computing system 100. The management 1030 resources may include administrative interfaces, orchestration frameworks, and automated scaling policies configured to manage deployment, performance, and availability of cloud-hosted workloads. In one or more embodiments, the management 1030 resources may further support coordination of data integration, analytical processing, simulation, and iterative refinement operations to maintain continuous and responsive execution of pathway-based health optimization processes.

In some embodiments, the cloud-based computing environment may further support privacy-preserving computation techniques, including secure aggregation, encrypted computation, or distributed learning mechanisms, to enable analysis of biologically-related data sets across organizational or jurisdictional boundaries. Such techniques may allow generation and refinement of pathway-based virtual representations while limiting exposure of sensitive patient data and maintaining compliance with applicable data protection requirements.

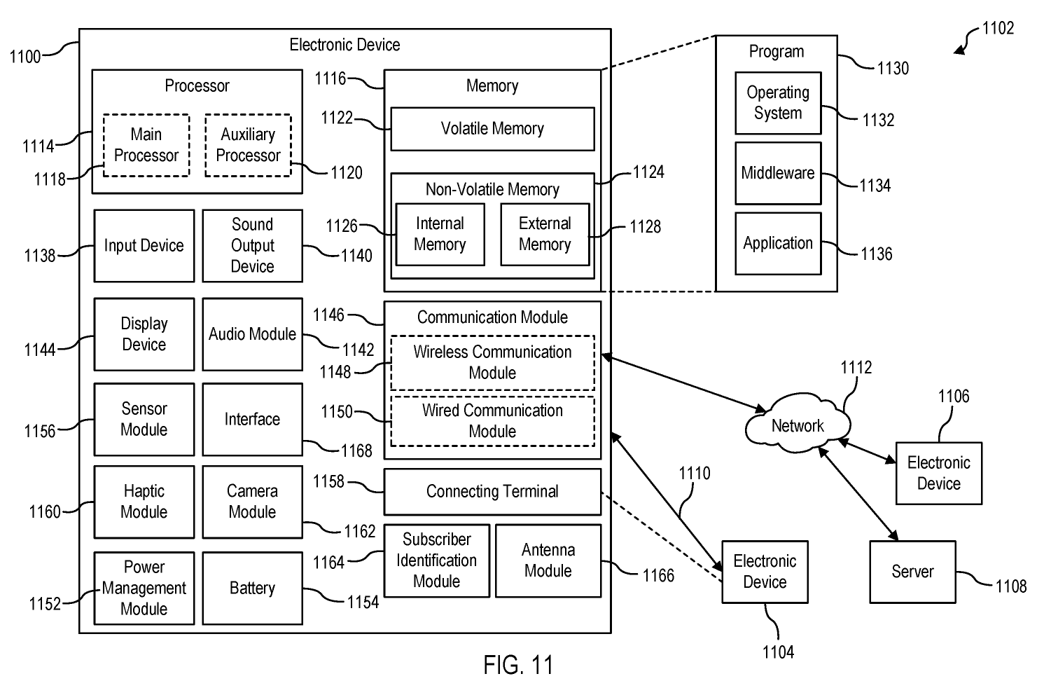

FIG. 10 therefore illustrates a cloud-based computing environment configured to support deployment, operation, and scaling of the system 200 and the computing system 100 for pathway-based health optimization. The arrangement shown in FIG. 10 demonstrates how the infrastructure 1004 resources, the platform 1012 resources, and the software 1020 resources cooperate to provide continuous data processing, pathway-based analysis, simulation, and secure delivery of health-related recommendations. By leveraging the cloud service provider 1002, distributed systems may execute pathway-based health optimization operations without reliance on local infrastructure. FIG. 11 sets forth a block diagram illustrating an example electronic device suitable for implementing one or more components of the system 200 or the computing system 100, providing the processing, storage, and communication resources required to execute the operations described throughout the present disclosure.

FIG. 11 is a block diagram of an electronic device 1100 in a network environment 1102 in accordance with embodiments of the present disclosure. The electronic device 1100 may operate independently or in conjunction with one or more other electronic devices 1104 and 1106, or a server 1108, through a first network 1110 (e.g., a short-range communication network) or a second network 1112 (e.g., a long-range communication network). The electronic device 1100 may correspond to, or include, the functional components of the computing system 100 or the system 200 described with reference to FIGS. 1 and 2. For example, the electronic device 1100 may execute one or more functionalities associated with at least the data integration layer 206, the processing layer 210, the digital twin agentic system 212, and the data lake house 214 to perform operations associated with pathway-based health optimization, including processing biologically-related data sets, generating a pathway-based virtual representation of a patient, conducting predictive risk assessment, simulating safety and efficacy models, iteratively refining biologically-related strategies, and generating and presenting health-related recommendations.

Referring to FIG. 11, the components of the electronic device 1100 illustrated therein will now be described in additional detail. The components of the electronic device 1100 may collectively enable execution of the systems and methods described throughout this disclosure, including at least processing 300 the one or more autonomously integrated biologically-related data sets, generating 302 the virtual representation of the patient, displaying 304 the one or more health-related recommendations, conducting 500 the one or more predictive risk assessments, simulating 600 the one or more safety and efficacy models, iteratively refining 700 the one or more strategies, executing 800 the real-time biological health state modeling, and providing 900 the internal anatomical visualization capabilities and the external form visualization capabilities associated with pathway-based health optimization. While particular components are shown in FIG. 11, additional or alternative components may be included in other embodiments, and the illustrated components may be implemented as discrete hardware modules, integrated circuits, software-executed components, or combinations thereof.

A processor 1114 may control overall operation of the electronic device 1100 and execute instructions stored in a memory 1116 to perform operations associated with pathway-based health optimization. The processor 1114 may include a main processor 1118 and an auxiliary processor 1120 that operate independently or cooperatively to manage computational, analytical, and communication tasks. The main processor 1118 may execute high-level operations of the system 200, including data integration, pathway-based analysis, predictive risk assessment, safety and efficacy simulation, and iterative refinement of biologically-related strategies. The auxiliary processor 1120 may perform supporting functions such as communication management, data synchronization with external systems, or background processing associated with monitoring incoming biologically-related data sets. In some embodiments, the auxiliary processor 1120 may continue to operate while the main processor 1118 is in a reduced-power state to maintain connectivity, receive updated data, or support continuous operation of pathway-based health optimization functions.

The memory 1116 may include both volatile memory 1122 and non-volatile memory 1124 configured to store data and instructions used by the processor 1114 during operation of the electronic device 1100. The non-volatile memory 1124 may include internal memory 1126 and external memory 1128 that store biologically-related data sets, pathway-based virtual representations, configuration parameters, and executable instructions associated with pathway-based health optimization. The memory 1116 may further store a program 1130 that includes an operating system 1132, middleware 1134, and one or more applications 1136 executed by the processor 1114 to perform operations described herein. In some embodiments, the memory 1116 may cache integrated biologically-related data sets, intermediate analytical results, predictive outputs, or visualization data to support efficient execution of at least processing 300 the one or more autonomously integrated biologically-related data sets, generating 302 the virtual representation of the patient, conducting 500 the one or more predictive risk assessments, simulating 600 the one or more safety and efficacy models, iteratively refining 700 the one or more strategies, executing 800 the real-time biological health state modeling, and providing 900 the internal anatomical visualization capabilities and the external form visualization capabilities.

An input device 1138 may receive user input, control commands, or external data during operation of the electronic device 1100. The input device 1138 may include a touchscreen, keyboard, mouse, microphone, or other input mechanisms that enable a user to provide configuration information, select operational modes, or input biologically-related data sets associated with a patient. In one or more embodiments, the input device 1138 may also receive data from external sources, such as connected monitoring devices or companion applications, for use in at least processing 300 the one or more autonomously integrated biologically-related data sets or generating 302 the virtual representation of the patient. The input device 1138 may further support voice-based or gesture-based interaction to facilitate use of the electronic device 1100 in clinical, research, or personal health environments.

A sound output device 1140 may output audio signals generated by the electronic device 1100 during operation. The sound output device 1140 may include one or more speakers, receivers, or other audio transducers configured to provide audible notifications, alerts, or feedback associated with pathway-based health optimization. In one or more embodiments, the sound output device 1140 may emit alerts corresponding to generation of health-related recommendations, detection of significant changes in biologically-related data sets, or completion of analytical operations such as predictive risk assessment or safety and efficacy simulation. The sound output device 1140 may operate in coordination with an audio module 1142 to support playback of audio prompts, spoken notifications, or other audible indicators that assist users in monitoring system status and interpreting generated outputs.

A display device 1144 may visually present information generated by the processor 1114 to a user of the electronic device 1100. The display device 1144 may include a flat-panel display, touchscreen display, or other visual output interface configured to render graphical user interfaces, charts, and visualizations associated with pathway-based health optimization. In one or more embodiments, the display device 1144 may present pathway-based virtual representations of a patient including internal anatomical visualization capabilities and external form visualization capabilities, as well as health-related recommendations generated during at least generating 302 the virtual representation of the patient and providing 900 the internal anatomical visualization capabilities and the external form visualization capabilities. The display device 1144 may further present alerts, status indicators, or interactive controls that allow a user to review analytical results, explore modeled outcomes, or interact with visualization outputs associated with the system 200.

A communication module 1146 may enable the electronic device 1100 to transmit and receive data through the first network 1110 or the second network 1112. The communication module 1146 may include a wireless communication module 1148 and a wired communication module 1150 that operate independently or cooperatively to support communication with external systems, devices, or networks. The wireless communication module 1148 may support technologies such as Wi-Fi, Bluetooth, near-field communication, or cellular connectivity to facilitate the exchange of biologically-related data sets, pathway-based virtual representations, and health-related recommendations between the electronic device 1100, the system 200, and cloud-based services. The wired communication module 1150 may support communication through physical interfaces such as USB or Ethernet to enable secure data transfer, synchronization, or configuration within clinical, research, or operational environments.

A power management module 1152 may regulate power distribution and consumption among the components of the electronic device 1100. The power management module 1152 may monitor voltage, current, and power usage associated with the processor 1114, the memory 1116, the communication module 1146, and other subsystems to maintain stable and efficient operation during the execution of pathway-based health optimization operations. The power management module 1152 may operate in conjunction with a battery 1154, which may supply electrical power to the electronic device 1100 through a rechargeable or replaceable power source. In one or more embodiments, the power management module 1152 may dynamically adjust power allocation based on computational workload, data transmission activity, or battery capacity to support continuous operation while conserving energy.

The battery 1154 may provide electrical power to one or more components of the electronic device 1100 under control of the power management module 1152. The battery 1154 may be implemented as a rechargeable battery or a replaceable power source configured to supply power required for continuous execution of pathway-based health optimization operations. In one or more embodiments, the battery 1154 may support sustained processing of biologically-related data sets, communication with external systems, and presentation of health-related recommendations. The battery 1154 may further support preservation of an operational state and stored data within the memory 1116 during temporary power interruptions to maintain continuity of system operation.

A sensor module 1156 may detect physiological, environmental, or operational conditions and generate corresponding signals for processing by the processor 1114. The sensor module 1156 may include one or more biosensors, motion sensors, temperature sensors, or other sensing elements configured to collect biologically-related data sets associated with a patient or operating conditions of the electronic device 1100. In one or more embodiments, the sensor module 1156 may acquire physiological measurements such as heart rate, activity level, or other biometric indicators for use during at least processing 300 of the one or more autonomously integrated biologically-related data sets and generating 302 the virtual representation of the patient. The sensor module 1156 may also detect environmental conditions or device status parameters that are incorporated into pathway-based health optimization analyses or used to support reliable operation of the electronic device 1100.

A connecting terminal 1158 may include one or more physical connectors configured to interface the electronic device 1100 with external equipment or peripheral devices. The connecting terminal 1158 may support wired communication standards such as USB, HDMI, or other connector types to enable data transfer, device configuration, diagnostic access, or charging. In one or more embodiments, the connecting terminal 1158 may facilitate connection of the electronic device 1100 to external monitoring devices, docking stations, or clinical systems to support synchronization of biologically-related data sets, retrieval of analytical results, or execution of pathway-based health optimization operations.

A haptic module 1160 may provide tactile feedback to a user of the electronic device 1100 during operation. The haptic module 1160 may include one or more actuators or vibration elements configured to generate physical sensations corresponding to alerts, notifications, or user interactions associated with pathway-based health optimization. In one or more embodiments, the haptic module 1160 may provide tactile alerts to indicate generation of health-related recommendations, detection of significant changes in biologically-related data sets, or completion of analytical operations such as predictive risk assessment or safety and efficacy simulation. The haptic module 1160 may operate in coordination with the display device 1144 and the sound output device 1140 to deliver multimodal feedback that enhances user awareness and interaction with the electronic device 1100.

A camera module 1162 may capture still images or video data during operation of the electronic device 1100. The camera module 1162 may include one or more image sensors and optical components configured to acquire visual information associated with the patient or the operating environment of the electronic device 1100. In one or more embodiments, the camera module 1162 may support capture of images or video used for documentation, remote consultation, or verification purposes related to pathway-based health optimization. The camera module 1162 may also operate in coordination with the processor 1114 and the communication module 1146 to transmit captured visual data to external systems or cloud-based services for further analysis or storage.

A subscriber identification module 1164 may store authentication credentials, user identification information, or subscription-related data used to authorize the electronic device 1100 for access to networks and services. The subscriber identification module 1164 may include a secure element, such as a SIM card, embedded SIM, or cryptographic processor, configured to support secure identification and authentication of the electronic device 1100 within the first network 1110 or the second network 1112. In one or more embodiments, the subscriber identification module 1164 may enable secure access to cloud-based services associated with pathway-based health optimization, restrict access to biologically-related data sets and health-related recommendations to authorized users, and support compliance with security or privacy requirements applicable to healthcare environments.

An antenna module 1166 may enable wireless transmission and reception of signals between the electronic device 1100 and external systems through the communication module 1146. The antenna module 1166 may include one or more antennas configured to support wireless communication protocols such as Wi-Fi, Bluetooth, cellular communication, or other radio-frequency technologies. In one or more embodiments, the antenna module 1166 may facilitate real-time communication with wearable sensors, external monitoring devices, or cloud-based computing environments that support pathway-based health optimization. The antenna module 1166 may be configured to maintain reliable connectivity and data transfer performance during continuous acquisition of biologically-related data sets and transmission of health-related recommendations.

An interface 1168 may support communication and data exchange between the electronic device 1100 and external peripherals, systems, or networks. The interface 1168 may include hardware and software components configured to facilitate input and output operations using wired or wireless communication protocols. In one or more embodiments, the interface 1168 may enable integration with external healthcare systems, data repositories, or third-party platforms to support exchange of biologically-related data sets, pathway-based virtual representations, and health-related recommendations. The interface 1168 may further support interoperability between the electronic device 1100, the system 200, and the computing system 100 to enable coordinated operation within distributed healthcare and research environments.

FIG. 11 therefore illustrates an example electronic device 1100 and network environment 1102 configured to execute the systems and methods for pathway-based health optimization described herein. The arrangement of components shown in FIG. 11 demonstrates how the system 200 and the computing system 100 may be implemented across mobile, clinical, or distributed computing environments to support processing of biologically-related data sets, generation of pathway-based virtual representations of patients, and presentation of health-related recommendations. The described configuration provides hardware and communication resources for executing at least the processing 300 of the one or more autonomously integrated biologically-related data sets, generating 302 the virtual representation of the patient, displaying 304 the one or more health-related recommendations, conducting 500 the one or more predictive risk assessments, simulating 600 the one or more safety and efficacy models, iteratively refining 700 the one or more strategies, executing 800 the real-time biological health state modeling, and providing 900 the internal anatomical visualization capabilities and the external form visualization capabilities, each of which are described throughout this disclosure. Collectively, FIGS. 1-11 illustrate a comprehensive computing and communication framework that supports secure, scalable, and intelligent pathway-based health optimization using integrated biologically-related data sets in accordance with embodiments of the present disclosure.

In one or more embodiments, the autonomous digital twin agentic system 212 implements comprehensive health state monitoring by integrating anatomical monitoring and functional monitoring of the patient. Anatomical monitoring may include real-time tracking of structural changes, dynamic updating of anatomical models, correlation of physical changes with health outcomes, visualization of treatment impacts on form, disease progression monitoring, surgical outcome prediction, age-related structural changes, or a combination thereof. Functional monitoring may include continuous biological pathway analysis, real-time physiological response tracking, and integration of multi-omics data.

In one or more embodiments, the systems and methods described herein improve the operation of a computing system itself by restructuring how biologically-related data sets are ingested, analyzed, and evaluated within the computing environment. Conventional healthcare computing systems rely on modality-specific pipelines, static feature extraction, or batch-oriented analytics that require repeated re-computation when new data is received, resulting in increased computational overhead, latency, and inconsistent analytical outcomes. In contrast, the disclosed pathway-based computational framework enables continuous, incremental updating of a virtual representation of a patient at a pathway level, such that newly received biologically-related data sets are integrated and evaluated without reprocessing the full data corpus. By maintaining pathway-level state, causal relationships, and spatiotemporal context within the virtual representation, the computing system reduces redundant computation, improves data locality, and enables predictive and simulation-based analysis to be executed with lower computational cost and improved stability. These improvements are realized at the level of data structures, processing flow, and system architecture, thereby providing a concrete technical improvement to the functioning of the computing system rather than a mere automation of existing clinical or analytical workflows.

In view of the explanations set forth above, at least one skilled in the art will recognize that embodiments of the present disclosure provide further significant technical and functional advantages over conventional healthcare computing systems. These advantages arise from the manner in which the disclosed systems integrate, model, and analyze biologically-related data sets within a pathway-based computational framework. For example, such advantages can also include, but are not limited to:

Enabling a unified, continuously adaptive analytical framework that overcomes fragmentation across clinical, molecular, physiological, and longitudinal data sources, thereby allowing biologically-related data sets to be evaluated in context rather than as isolated measurements.

Providing a computational digital twin that represents patient biology at a pathway level, allowing system behavior to reflect underlying biological mechanisms and interactions rather than surface-level correlations or static indicators.

Improving the reliability and interpretability of health-related outputs by grounding analysis in pathway-level relationships, biological variation, and spatiotemporal behavior, which reduces dependence on purely correlative or retrospective analytical techniques.

Supporting forward-looking assessment of patient health states through predictive modeling that anticipates risk, response, or progression based on evolving biological conditions, rather than reacting only after changes occur.

Increasing robustness and safety of generated outputs by enabling simulation and evaluation of potential outcomes prior to presentation, allowing recommendations to be assessed against modeled biological behavior and constraints before use.

Allowing health-related strategies to evolve dynamically through iterative refinement informed by new data, predicted outcomes, and modeled responses, thereby accommodating biological variability and temporal change across individual patients.

Enhancing human interpretability and system usability by providing internal anatomical and external form visualizations that convey biological state and pathway behavior in a manner that supports expert review, oversight, and decision making.

Collectively, these advantages allow the disclosed systems to operate in a manner that is more adaptive, biologically informed, and computationally effective than conventional health data processing approaches, while maintaining scalability across diverse data sources, deployment environments, and application contexts.

Exemplary embodiments of the present invention are described largely in the context of a fully functional computer system for encoding an object stream, as is described herein. Readers of skill in the art will recognize, however, that the present invention also may be embodied in a computer program product disposed upon computer readable storage media for use with any suitable data processing system. Such computer readable storage media may be any storage medium for machine-readable information, including magnetic media, optical media, or other suitable media. Examples of such media include magnetic disks in hard drives or diskettes, compact disks for optical drives, magnetic tape, and others as will occur to those of skill in the art. Persons skilled in the art will immediately recognize that any computer system having suitable programming means will be capable of executing the steps of the method of the invention as embodied in a computer program product. Persons skilled in the art will recognize also that, although some of the exemplary embodiments described in this specification are oriented to software installed and executing on computer hardware, nevertheless, alternative embodiments implemented as firmware or as hardware are well within the scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the "C" programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Advantages and features of the present disclosure can be further described by the following statements:

Statement 1. A method, comprising: processing, by a computing device, one or more autonomously integrated biologically-related data sets corresponding to a patient; generating, by the computing device and in response to processing the one or more autonomously integrated biologically-related data sets, a virtual representation of the patient via one or more biological pathways; and displaying, by the computing device, one or more health-related recommendations generated based on an analysis of the one or more biologically-related data sets using the virtual representation of the patient.

Statement 2. The method of the statement above, wherein autonomously integrating the one or more biologically-related data sets comprises: executing, by the computing device, a biological variation analysis on the one or more biologically-related data sets received from the patient including at least one of a trend analysis or a variation detection; and identifying, by the computing device, one or more spatiotemporal patterns corresponding to the one or more biologically-related data sets identified in response to executing the biological variation analysis.

Statement 3. The method of any combination of one or more of the statements above, wherein at least one of the one or more spatiotemporal patterns or one or more response patterns are autonomously integrated within the one or more biological pathways in real-time and based on one or more genetic influences associated with the patient.

Statement 4. The method of any combination of one or more of the statements above, further comprising: conducting, by the computing device using one or more multi-factor analyses, one or more predictive risk assessments associated with the one or more biologically-related data sets.

Statement 5. The method of any combination of one or more of the statements above, further comprising: simulating, by the computing device, one or more safety and efficacy models associated with the one or more health-related recommendations.

Statement 6. The method of any combination of one or more of the statements above, further comprising: iteratively refining, by the computing device, one or more strategies for adjusting one or more biologically-related solutions corresponding to the one or more health-related recommendations.

Statement 7. The method of any combination of one or more of the statements above, wherein generating the virtual representation of the patient comprises: executing, by the computing device, real-time biological health state modeling including an internal anatomical model of the patient and an external form representation of the patient.

Statement 8. The method of any combination of one or more of the statements above, wherein the one or more health-related recommendations are displayed on a display screen as a graphical user interface, and wherein displaying the one or more health-related recommendations comprises: providing, by the computing device and to a user, internal anatomical visualization capabilities associated with the patient and external form visualization capabilities associated with the patient.

Statement 9. A system comprising: a memory; and a processing device, operatively coupled to the memory, the processing device configured to: process one or more autonomously integrated biologically-related data sets corresponding to a patient; generate, in response to processing the one or more autonomously integrated biologically-related data sets, a virtual representation of the patient via one or more biological pathways; and display one or more health-related recommendations generated based on an analysis of the one or more biologically-related data sets using the virtual representation of the patient.

Statement 10. The system of any combination of one or more of the statements above, wherein the processing device configured to autonomously integrate the one or more biologically-related data sets is further configured to: execute a biological variation analysis on the one or more biologically-related data sets received from the patient including at least one of a trend analysis or a variation detection; and identify one or more spatiotemporal patterns corresponding to the one or more biologically-related data sets identified in response to executing the biological variation analysis.

Statement 11. The system of any combination of one or more of the statements above, wherein at least one of the one or more spatiotemporal patterns or one or more response patterns are autonomously integrated within the one or more biological pathways in real-time and based on one or more genetic influences associated with the patient.

Statement 12. The system of any combination of one or more of the statements above, wherein the processing device is further configured to: conduct, using one or more multi-factor analyses, one or more predictive risk assessments associated with the one or more biologically-related data sets.

Statement 13. The system of any combination of one or more of the statements above, wherein the processing device is further configured to: simulate one or more safety and efficacy models associated with the one or more health-related recommendations.

Statement 14. The system of any combination of one or more of the statements above, wherein the processing device is further configured to: iteratively refine one or more strategies for adjusting one or more biologically-related solutions corresponding to the one or more health-related recommendations.

Statement 15. The system of any combination of one or more of the statements above, wherein the processing device configured to generate the virtual representation of the patient is further configured to: execute real-time biological health state modeling including an internal anatomical model of the patient and an external form representation of the patient.

Statement 16. The system of any combination of one or more of the statements above, wherein the one or more health-related recommendations are displayed on a display screen as a graphical user interface, and wherein the processing device configured to display the one or more health-related recommendations is further configured to: provide, to a user, internal anatomical visualization capabilities associated with the patient and external form visualization capabilities associated with the patient.

Statement 17. A non-transitory computer-readable media storing processor-executable instructions that, when executed by at least one processor, cause the at least one processor to: process one or more autonomously integrated biologically-related data sets corresponding to a patient; generate, in response to processing the one or more autonomously integrated biologically-related data sets, a virtual representation of the patient via one or more biological pathways; and display one or more health-related recommendations generated based on an analysis of the one or more biologically-related data sets using the virtual representation of the patient.

Statement 18. The computer-readable media of any combination of one or more of the statements above, wherein the at least one processor caused to autonomously integrate the one or more biologically-related datasets is further caused to: execute a biological variation analysis on the one or more biologically-related data sets received from the patient including at least one of a trend analysis or a variation detection; and identify one or more spatiotemporal patterns corresponding to the one or more biologically-related data sets identified in response to executing the biological variation analysis, wherein at least one of the one or more spatiotemporal patterns or one or more response patterns are autonomously integrated within the one or more biological pathways in real-time and based on one or more genetic influences associated with the patient.

Statement 19. The computer-readable media of any combination of one or more of the statements above, wherein the at least one processor caused to generate the virtual representation of the patient is further caused to: execute real-time biological health state modeling including an internal anatomical model of the patient and an external form representation of the patient.

Statement 20. The computer-readable media of any combination of one or more of the statements above, wherein the one or more health-related recommendations are displayed on a display screen as a graphical user interface, and wherein the at least one processor caused to display the one or more health-related recommendations is further caused to: provide, to a user, internal anatomical visualization capabilities associated with the patient and external form visualization capabilities associated with the patient.

It will be understood from the foregoing description that modifications and changes may be made in various embodiments of the present invention without departing from its true spirit. The descriptions in this specification are for purposes of illustration only and are not to be construed in a limiting sense. The scope of the present invention is limited only by the language of the following claims.

What is claimed is:

1. A method, comprising:
processing, by a computing device, one or more autonomously integrated biologically-related data sets corresponding to a patient;
generating, by the computing device and in response to processing the one or more autonomously integrated biologically-related data sets, a virtual representation of the patient via one or more biological pathways; and
displaying, by the computing device, one or more health-related recommendations generated based on an analysis of the one or more biologically-related data sets using the virtual representation of the patient.

2. The method of claim 1, wherein autonomously integrating the one or more biologically-related data sets comprises:
executing, by the computing device, a biological variation analysis on the one or more biologically-related data sets received from the patient including at least one of a trend analysis or a variation detection; and
identifying, by the computing device, one or more spatiotemporal patterns corresponding to the one or more biologically-related data sets identified in response to executing the biological variation analysis.

3. The method of claim 2, wherein at least one of the one or more spatiotemporal patterns or one or more response patterns are autonomously integrated within the one or more biological pathways in real-time and based on one or more genetic influences associated with the patient.

4. The method of claim 1, further comprising:
conducting, by the computing device using one or more multi-factor analyses, one or more predictive risk assessments associated with the one or more biologically-related data sets.

5. The method of claim 1, further comprising:
simulating, by the computing device, one or more safety and efficacy models associated with the one or more health-related recommendations.

6. The method of claim 1, further comprising:
iteratively refining, by the computing device, one or more strategies for adjusting one or more biologically-related solutions corresponding to the one or more health-related recommendations.

7. The method of claim 1, wherein generating the virtual representation of the patient comprises:
executing, by the computing device, real-time biological health state modeling including an internal anatomical model of the patient and an external form representation of the patient.

8. The method of claim 1, wherein the one or more health-related recommendations are displayed on a display screen as a graphical user interface, and wherein displaying the one or more health-related recommendations comprises:
providing, by the computing device and to a user, internal anatomical visualization capabilities associated with the patient and external form visualization capabilities associated with the patient.

9. A system comprising:
a memory; and
a processing device, operatively coupled to the memory, the processing device configured to:
process one or more autonomously integrated biologically-related data sets corresponding to a patient;
generate, in response to processing the one or more autonomously integrated biologically-related data sets, a virtual representation of the patient via one or more biological pathways; and
display one or more health-related recommendations generated based on an analysis of the one or more biologically-related data sets using the virtual representation of the patient.

10. The system of claim 9, wherein the processing device configured to autonomously integrate the one or more biologically-related data sets is further configured to:
execute a biological variation analysis on the one or more biologically-related data sets received from the patient including at least one of a trend analysis or a variation detection; and
identify one or more spatiotemporal patterns corresponding to the one or more biologically-related data sets identified in response to executing the biological variation analysis.

11. The system of claim 10, wherein at least one of the one or more spatiotemporal patterns or one or more response patterns are autonomously integrated within the one or more biological pathways in real-time and based on one or more genetic influences associated with the patient.

12. The system of claim 9, wherein the processing device is further configured to:
conduct, using one or more multi-factor analyses, one or more predictive risk assessments associated with the one or more biologically-related data sets.

13. The system of claim 9, wherein the processing device is further configured to:
simulate one or more safety and efficacy models associated with the one or more health-related recommendations.

14. The system of claim 9, wherein the processing device is further configured to:
iteratively refine one or more strategies for adjusting one or more biologically-related solutions corresponding to the one or more health-related recommendations.

15. The system of claim 9, wherein the processing device configured to generate the virtual representation of the patient is further configured to:
execute real-time biological health state modeling including an internal anatomical model of the patient and an external form representation of the patient.

16. The system of claim 9, wherein the one or more health-related recommendations are displayed on a display screen as a graphical user interface, and wherein the processing device configured to display the one or more health-related recommendations is further configured to:
provide, to a user, internal anatomical visualization capabilities associated with the patient and external form visualization capabilities associated with the patient.

17. A non-transitory computer-readable media storing processor-executable instructions that, when executed by at least one processor, cause the at least one processor to:
process one or more autonomously integrated biologically-related data sets corresponding to a patient;
generate, in response to processing the one or more autonomously integrated biologically-related data sets, a virtual representation of the patient via one or more biological pathways; and
display one or more health-related recommendations generated based on an analysis of the one or more biologically-related data sets using the virtual representation of the patient.

18. The computer-readable media of claim 17, wherein the at least one processor caused to autonomously integrate the one or more biologically-related datasets is further caused to:
execute a biological variation analysis on the one or more biologically-related data sets received from the patient including at least one of a trend analysis or a variation detection; and
identify one or more spatiotemporal patterns corresponding to the one or more biologically-related data sets identified in response to executing the biological variation analysis, wherein at least one of the one or more spatiotemporal patterns or one or more response patterns are autonomously integrated within the one or more biological pathways in real-time and based on one or more genetic influences associated with the patient.

19. The computer-readable media of claim 17, wherein the at least one processor caused to generate the virtual representation of the patient is further caused to:
execute real-time biological health state modeling including an internal anatomical model of the patient and an external form representation of the patient.

20. The computer-readable media of claim 17, wherein the one or more health-related recommendations are displayed on a display screen as a graphical user interface, and wherein the at least one processor caused to display the one or more health-related recommendations is further caused to:
provide, to a user, internal anatomical visualization capabilities associated with the patient and external form visualization capabilities associated with the patient.
